SEQUENCE LISTING (5 sequences)

SEQ ID NO: 1 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 2 - protein, 16 aa, Homo sapiens
MVSHHNLTTGATLINE

SEQ ID NO: 3 - protein, 8 aa, Homo sapiens
NLFLNHSE

SEQ ID NO: 4 - protein, 5 aa, Homo sapiens
NATAK

SEQ ID NO: 5 - protein, 11 aa, Homo sapiens
VVLHPNYSQVD

(19) United States
(12) Patent Application Publication
(10) Pub. No.: US 2025/0138017 A1
(43) Pub. Date: May 1, 2025

(54) GLYCAN STRUCTURES OF HAPTOGLOBIN AS A BIOMARKER OF HEPATOCELLULAR CARCINOMA

(71) Applicant: Roche Diagnostics Operations, Inc., Indianapolis, IN (US)

(72) Inventors: Vinzent Rolny, München (DE); Holger Busskamp, München (DE); Konstantin Kroeniger, Oberammergau (DE); Mahdokht Kohansal Nodehi, München (DE); Magdalena Swiatek-de Lange, Penzberg (DE); Gloria Tabares, Muenchen (DE)

(21) Appl. No.: 18/930,774

(22) Filed: Oct. 29, 2024

Related U.S. Application Data
(63) Continuation of application No. PCT/EP2023/061066, filed on Apr. 27, 2023.

(30) Foreign Application Priority Data
Apr. 29, 2022 (EP) 22170949.6

(51) Int. Cl. G01N 33/574; G01N 33/68

(52) U.S. Cl. CPC G01N 33/57488; G01N 33/6848; G01N 2440/38

(57) ABSTRACT

The present invention relates to in vitro methods for aiding in the detection of hepatocellular carcinoma (HCC) in a subject. The method may comprise determining the amount of one or more N-glycan structure attached to haptoglobin (i.e. of the β-chain of haptoglobin having the sequence given in SEQ ID NO: 1) in a sample obtained from said subject and comparing the amount of said one or more glycan structure to a reference amount of said one or more glycan structure, wherein an altered amount of said one or more glycan structure in said patient sample relative to the reference amount of said one or more glycan structure is indicative for HCC. Further, the present invention relates to the use of one or more glycan structure attached to haptoglobinor of a glycopeptide derived from haptoglobin in combination with AFP and/or PIVKA-II in the detection of HCC.

### FIELD OF THE INVENTION

The present invention relates to in vitro methods for aiding in the detection of hepatocellular carcinoma (HCC) in a subject. The method may comprise determining the amount of one or more N-glycan structure attached to haptoglobin (i.e. of the β-chain of haptoglobin having the sequence given in SEQ ID NO: 1) in a sample obtained from said subject and comparing the amount of said one or more glycan structure to a reference amount of said one or more glycan structure, wherein an altered amount of said one or more glycan structure in said patient sample relative to the reference amount of said one or more glycan structure is indicative for HCC. Further, the present invention relates to the use of one or more glycan structure attached to haptoglobinor of a glycopeptide derived from haptoglobin in combination with AFP and/or PIVKA-II in the detection of HCC.

### BACKGROUND OF THE INVENTION

Liver cancer is the seventh most common cancer and the second cause of death from cancer worldwide. The incidence rate and mortality rate were 10.1 and 9.5, respectively, per 100,000 persons in 2012.

Hepatocellular carcinoma (HCC) is the major histologic type among primary liver cancers occurring worldwide, accounting for 70% to 85% of the total burden. It is known, that underlying liver diseases such as liver fibrosis and cirrhosis are the main risk factors for the development of HCC. HCC can be treated by resection, liver transplantation, or local ablation with radiofrequency for patients diagnosed at an early stage.

The 5-year survival rate of the HCC patients may be as high as 70% if this malignancy is diagnosed in an early stage. However, the 5-year survival rate of the HCC patients decreases significantly the later the disease is diagnosed and drops to only 15%, if HCC is diagnosed in the late stage of disease (Tsuchiya N, Sawada Y, Endo I, et al. Biomarkers for the early diagnosis of hepatocellular carcinoma. World J Gastroenterol. 2015; 21(37):10573-83; Siegel R, Naishadham D, Jemal A. Cancer statistics, 2013. CA: A Cancer Journal for Clinicians. 2013; 63(1):11-30). Due to lack of symptoms more than 60% of HCC patients are diagnosed at late stage when the metastasis has already occurred (Altekruse S F, McGlynn K A, Reichman M E. Hepatocellular carcinoma incidence, mortality, and survival trends in the United States from 1975 to 2005. Journal of clinical oncology: official journal of the American Society of Clinical Oncology. 2009; 27(9):1485-91). Therefore, development of a noninvasive early HCC diagnosis test HCC is essential to reduce the death rate and increase the efficiency/success rate of HCC treatment.

The most common methods for diagnosis of HCC are ultrasound detection, imaging techniques such as computed axial tomography (CAT scan) or magnetic resonance imaging (MRI) and serological biomarkers. However, the ultra-sound detection has poor prognosis value, as it requires at least a 2 cm tumor mass and the imaging techniques have poor sensitivity of a per lesion basis and high costs. On the other hand, a lot of focus has been put to discover new blood biomarker that can be used in surveillance programs for early detection of HCC in high-risk patients in recent years (Yang J D. Detect or not to detect very early stage hepatocellular carcinoma? The western perspective. Clin Mol Hepatol. 2019; 25(4):335-43).

Several current medical guidelines recommend surveillance of high-risk patients using ultrasound, or other imaging modalities, every 6 months. However, limitations of the imaging approach include poor sensitivity for early stage tumors, operator-dependency, and lower quality in patients with obesity or non-alcoholic steatohepatitis. The adjunctive use of the tumor biomarker AFP may improve detection rates, but has limited sensitivity for detecting small tumors.

Haptoglobin (Hp) is an acute-phase glycoprotein with four N-glycosylation sites. Like many other inflammation markers and like many other tumor-related biomarkers it has been occasionally mentioned that Hp might be a marker candidate in the field of HCC (Tai C-S, Lin Y-R, Teng T-H, Lin P-Y, Tu S J, Chou C-H, Huang Y-R, Huang W-C, Weng S-L, Huang H D, Chen Y-L, Chen W L. Haptoglobin expression correlates with tumor differentiation and five-year overall survival rate in hepatocellular carcinoma. PLoS ONE 2017; 12(2): e0171269). Aberrant glycosylation of serum haptoglobin, especially on the glycosite Asn241, has been recently shown to correlate with the occurrence of the gastric cancer (Jeong S, Kim U, Oh M J, Nam J, Park S H, Choi Y J, Lee D H, Kim J, An HJ. Detection of Aberrant Glycosylation of Serum Haptoglobin for Gastric Cancer Diagnosis Using a Middle-Up-Down Glycoproteome Platform. J. Pers. Med. 2021; 11: 575). Specific glycosylation changes of haptoglobin has been also reported for other types of cancers, including liver, breast, lung, prostate, pancreatic an colon cancer (Oh M J, Lee S H, Kim U, An HJ. In-depth investigation of altered glycosylation in human haptoglobin associated cancer by mass spectrometry. Mass Spec Rev. 2021; 1-23).

Ramachandran P. et al. (2022) recently described serum glycoprotein markers in nonalcoholic steatohepatitis and hepatocellular carcinoma. The study also included mass-spectrometry based glycan evaluation at amino acid residue positions 184, 207, 211, and 241 of haptoglobin (Ramachandran, Gege Xu, Hector H. Huang, Rachel Rice, Bo Zhou, Klaus Lindpaintner, and Daniel Serie Journal of Proteome Research 2022 21 (4), 1083-1094. DOI: 10.1021/acs.jproteome.1c00965).

Alpha-fetoprotein (AFP) is the best-established blood biomarker of HCC. However, even AFP demonstrates suboptimal sensitivity and specificity for early detection of the HCC. In addition, it has been reported that the AFP level might be elevated falsely in patients with chronic hepatitis or cirrhosis without HCC.

AFP is a glycoprotein and various glycosylated forms of AFP have been described. Lectins can be used in the analysis of glycoproteins. By using the selective binding capacity of a lectin to the sugar chain structure of a glycoprotein it is possible to separate and concentrate the marker glycoprotein fraction(s) having a specific sugar chain structure. In the case of AFP, the lectin derived from Lens culinaris agglutinin-A (LCA) has been widely used. The Lens culinaris agglutinin (LCA)-reactive fraction of α-fetoprotein (AFP-L3) is specifically increased in patients with HCC. Many attempts have been made to specifically measure AFP-L3, e.g., by affinity electrophoresis using LCA, lectin-based ELISAs or by antibodies specifically binding the L3-form of AFP.

High-speed/high-sensitivity qualitative/quantitative analysis using a mass spectrometer is also used for the analysis of glycoproteins. Multiple reaction monitoring mass spectrometry (MRM) facilitates the quantification of peptides produced from protein hydrolysis and is quite reliable. The parallel reaction monitoring (PRM) technique uses a mass spectrometer equipped with a trap and a time-of-flight mass analyzer, so that a product ion spectrum of the peptide can be obtained, allowing quantitative and qualitative analysis of the peptide simultaneously. This method can also analyze trace glycoproteins that exhibit low signals with high reproducibility and good sensitivity.

Methods for analyzing a specific sugar chain using a mass spectrometer include methods based on the analysis of the sugar chain(s) separated from glycoproteins and methods based on the analysis of the sugar chain bound peptides, i.e. glycopeptide(s).

Sugar chains bound at one and the same amino acid position of a protein may have various structures and exhibit heterogeneity. It is also known that sugar structures may vary depending on the amino acid position at which the sugar chain is located.

Based on MRM-MS analysis Kim and coworkers demonstrated that the deglycopeptide of α-fetoprotein can distinguish cancer status between normal subjects and hepatocellular carcinoma patients better than the nonglycopeptide (Kim H, Kim K, Jin J, Park J, Yu S J, Yoon J-H, Kim Y. Measurement of Glycosylated Alpha-Fetoprotein Improves Diagnostic Power over the Native Form in Hepatocellular Carcinoma. PLOS One, 2014, 9: e110366). In EP 3 415 918 various glycopeptides of AFP-L3 have been analyzed by mass spectrometry and e.g. used to compare a sample obtained from a patient with HCC to a sample obtained from a subject having liver cirrhosis or hepatitis.

Protein induced by vitamin K absence/antagonist-II (PIVKA-II), also known as des-gamma-carboxy-prothrombin (DCP) is an abnormal form of prothrombin protein elevated in HCC patients and used as an alternative HCC biomarker individually or in combination with AFP. Prothrombin has 10 potential gamma-carboxylation sites and various forms of PIVKA-II with different levels of under-carboxylation are present in the circulation. Different assays for PIVKA-II may detect a different set of PIVKA-II-forms and the specificity/sensitivity of PIVKA-II might be variable depending on the assay used and of limited utility in the detection of early stage HCC.

Up to date, the international guidelines lack consensus on the use of AFP-L3 and PIVKA-II or alternative serum biomarkers for surveillance and diagnosis of at-risk patients. Yet, many recent publications clearly point out that scores comprising the input variables gender, age, AFP and descarboxyprothrombin (DCP=PIVKA-II) (=GAAD-score) or gender, age, AFP, AFP-L3 and descarboxyprothrombin (DCP=PIVKA-II) (=GALAD-score) can and will improve the results of HCC-screening efforts.

Nonetheless, there is a tremendous need to further improve the in vitro methods aiding in the detection of HCC.

Surprisingly, novel glycan structures on haptoglobin were discovered which are of high diagnostics value, e.g. in the early detection of HCC. Combination of these glycan structures with established reference protein biomarkers (e.g. AFP, PIVKA-II) improves their clinical utility even further.

### SUMMARY OF THE INVENTION

The present inventors have found that the analysis of glycan structure(s) attached to the β-chain of haptoglobin can overcome some of the problems with current in vitro diagnostic methods aiding in the detection of HCC.

In particular, the present inventors have identified glycan structures at position N184 of the β-chain of haptoglobin (i.e. at the amino acid asparagine in position 184 of SEQ ID NO:1) that are good biomarkers for detecting HCC, especially also early HCC.

In a first aspect, the present invention relates to an in vitro method for aiding in the detection of hepatocellular carcinoma (HCC) in a subject comprising the steps of: a) determining the amount of one or more glycan structure at position N184 of haptoglobin (i.e. of the β-chain of haptoglobin having the sequence given in SEQ ID NO: 1) in a sample obtained from said subject and b) comparing the amount of said one or more glycan structure detected in a) to a reference amount of said one or more glycan structure, wherein an altered amount of said one or more glycan structure in said patient sample relative to the reference amount of said one or more glycan structure is indicative for HCC.

In a specific embodiment, the one or more glycan structure attached to N184 is the glycan structure HexNAc(5)Hex(6)Fuc(1)NeuAc(2).

In a second aspect, herein provided is an in vitro method for aiding in the detection of HCC (e.g. early HCC) in a subject comprising the steps of: a) determining the amount of the N-glycan structure HexNAc(5)Hex(6)Fuc(1)NeuAc(2) at position N184 of haptoglobin (i.e. of the β-chain of haptoglobin having the sequence given in SEQ ID NO: 1) in a sample obtained from said subject and b) (i) comparing the amount of said N-glycan structure determined in a) to a reference amount of said N-glycan structure, wherein an altered amount of HexNAc(5)Hex(6)Fuc(1)NeuAc(2) at position N184 of haptoglobin in said subject's sample relative to the reference amount of this N-glycan structure is indicative for HCC (e.g. early HCC); or (ii) determining a score for detecting HCC (e.g. early HCC) taking into account the amount of HexNAc(5)Hex(6)Fuc(1)NeuAc(2) at position N184 of haptoglobin determined in a) and comparing the determined score for detecting HCC (e.g. early HCC) to a reference value for said score indicative for HCC (e.g. early HCC).

In a third aspect, the present invention relates to an isolated glycopeptide having a peptide part and a N-glycan part, wherein the peptide part comprises or consists of the amino acid sequence MVSHHNLTTGATLINE (SEQ ID NO: 2) and wherein the N-glycan part is HexNAc(5)Hex(6)Fuc(1)NeuAc(2), and wherein the N-glycan part is attached to the N in position 6 of SEQ ID NO: 2. In embodiments, the methionine in position 1 of SEQ ID NO: 2 may be oxidized.

In a specific embodiment of the third aspect of the present disclosure, herein provided is a gylcopeptide of Formula 1:

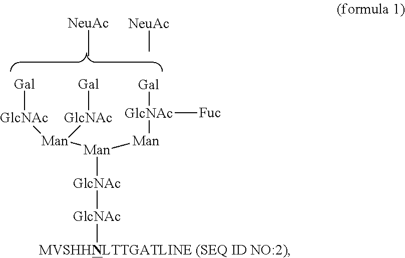

wherein GlcNAc means N-acetylglucosamine, Man means Mannose, Gal means Galactose, Fuc means Fucose and NeuAc means N-Acetyl-neuraminic acid (sialic acid). Lines represent covalent bonds. The glycan in the glycopeptide is covalently attached as N-glycan via the indicated GlcNac of the glycan to the side chain of an asparagine corresponding to N184 of haptoglobin. (i.e. N184 of the β-chain of haptoglobin of SEQ ID NO:1). In embodiments, the methionine in position 1 of SEQ ID NO: 2 may be oxidized.

The peptide comprised in this glycopeptide has the amino acid sequence as depicted in SEQ ID NO: 2. An alternative graphical presentation of the glycopeptide with the same structure as presented in formula 1, above, is presented in FIG. 2, right panel.

In a fourth aspect, the present invention relates to the use of the glycopeptide of the third aspect (e.g. of Formula 1, see above) in the detection of HCC.

According to a fifth aspect of the present disclosure, provided is a method for detecting a glycan structure at position N184 of haptoglobin (i.e. of the β-chain of haptoglobin having the sequence given in SEQ ID NO: 1), the method comprising the steps of: a) purifying haptoglobin from a sample to be analyzed; b) digesting the haptoglobin obtained in step a) by GluC and trypsin, and c) detecting the glycopeptide comprising position N184 of haptoglobin with the glycan structure attached thereto generated in b), thereby detecting the glycan structures at position N184.

In an sixth aspect, the present disclosure relates to an (in vitro) method for aiding in the detection of HCC (e.g. early HCC) in a subject comprising the steps of: a) determining the amount of a N-glycan structure at position N184 of haptoglobin (i.e. of the β-chain of haptoglobin having the sequence given in SEQ ID NO: 1) in a sample obtained from said subject, b) determining the amount of a HCC biomarker (e.g. the amount of AFP and/or the amount of PIVKA) other than the N-glycan structure at position N184 in a) in a sample obtained from said subject, c) combining the amounts determined in a) and b) to a combined value (e.g. a score for detecting HCC, e.g. early HCC) and comparing the combined value to a reference value for said combined value, wherein an altered combined value is indicative for HCC (e.g. early HCC).

In a seventh aspect, provided herein is a computer-implemented method for aiding in the detection of HCC (e.g. early HCC) in a subject, said method comprising:
a) receiving data comprising the amount of a glycan structure at position N184 of haptoglobin (i.e. of the β-chain of haptoglobin having the sequence given in SEQ ID NO: 1) in a sample obtained from said subject; and
b) i) comparing the amount of the glycan structure received in a) to a reference amount of said N-glycan structure at position N184 of haptoglobin, wherein an altered amount of the N-glycan structure in said patient sample relative to the reference amount of said one or more glycan structure is indicative for HCC; or (ii) calculating a score for detecting HCC (e.g. early HCC) taking into account the amount of said glycan structure at position N184 of haptoglobin received in a) and comparing the calculated score for detecting HCC (e.g. early HCC) to a reference value for said score indicative for HCC (e.g. early HCC).

In an eighth aspect, herein provided is a computer-implemented method for aiding in the detection of HCC (e.g. early HCC) in a subject, said method comprising:
a) receiving data comprising the amount of one or more glycan structure (e.g. HexNAc(5)Hex(6)Fuc(1)NeuAc(2)) at position N184 of haptoglobin (i.e. of the β-chain of haptoglobin having the sequence given in SEQ ID NO: 1) in a sample obtained from said subject
b) receiving data comprising the amount of PIVKA-II and/or AFP in the sample obtained from said subject,
c) calculating a score taking into account the amounts determined in a) and b); and
d) comparing the score calculated in c) to a reference value of said score indicative for HCC (e.g. early HCC).

Also provided herein is a computer program product comprising instructions which, when the program is executed by a computer, cause the computer to carry out the computer-implemented method according to the seventh or eighth aspect.

Further, provided is a computer-readable medium comprising instructions which, when executed by a computer, cause the computer to carry out the computer-implemented method according to the seventh or eighth aspect.

Also provided is a data processing system comprising a receiving unit configured to receiving data as defined in the seventh or eighth aspect; and a processing unit configured to perform the calculation and/or comparison step and/or any determination/assessment step in the seventh or eighth aspect; and optionally an outputting unit configured to output the assessment results.

### DETAILED DESCRIPTION OF THE INVENTION

In the following, the elements of the present invention will be described. These elements are listed with specific aspects and embodiments, however, it should be understood that they may be combined in any manner and in any number to create additional aspect and embodiments.

In a first aspect, the present invention relates to an in vitro method for aiding in the detection of hepatocellular carcinoma (HCC) in a subject comprising the steps of: a) determining the amount of one or more glycan structure at position N184 of haptoglobin (i.e. of the β-chain of haptoglobin having the sequence given in SEQ ID NO: 1) in a sample obtained from said subject and b) comparing the amount of said one or more glycan structure determined in a) to a reference amount of said glycan structure, wherein an altered amount of said one or more glycan structure in said patient sample relative to the reference amount of said one or more glycan structure is indicative for HCC.

In embodiments of the first aspect of the invention provided is an in vitro method for aiding in the detection of hepatocellular carcinoma (HCC) in a subject comprising the steps of: a) determining the amount of a glycan structure at position N184 of haptoglobin (i.e. of the β-chain of haptoglobin having the sequence given in SEQ ID NO: 1) in a sample obtained from said subject and b) comparing the amount of said glycan structure determined in a) to a reference amount of said glycan structure, wherein an altered amount of said glycan structure in said patient sample relative to the reference amount of said glycan structure is indicative for HCC.

The method of the first aspect of the invention is based on the finding that the glycosylation pattern at position 184 of the β-chain of haptoglobin can be used in the detection of HCC (e.g. early HCC). As demonstrated in the appended Examples, the glycan structures at this position in samples from patients with HCC (e.g. early HCC) show a characteristic pattern, e.g. as compared to reference samples, as well as compared to samples from patients with liver cirrhosis.

In embodiments of the first aspect of the invention, the determined amount(s) of the one or more glycan structures at position N184 of haptoglobin may not each be compared to a reference amount for said respective glycan structures but may instead a score for determining HCC (e.g. early HCC) taking into account the determined amount(s) of the one or more glycan structures at position N184 of haptoglobin may be calculated. Said score may be compared to a respective reference value for said score which is decisive for the presence HCC (e.g. early HCC).

Accordingly, herein provided is an in vitro method for aiding in the detection of hepatocellular carcinoma (HCC) in a subject comprising the steps of: a) determining the amount of a glycan structure at position N184 of haptoglobin (i.e. of the β-chain of haptoglobin having the sequence given in SEQ ID NO: 1) in a sample obtained from said subject and b) determining a score for detecting HCC (e.g. early HCC) taking into account the amount of said glycan structure determined in a); and c) comparing the determined score for detecting HCC (e.g. early HCC) to a reference value indicative for HCC (e.g. early HCC). Based on the comparison the presence or absence of HCC (e.g. early HCC) may be determined.

In embodiments, the glycan structure at position N184 of haptoglobin (i.e. of the β-chain of haptoglobin having the sequence given in SEQ ID NO: 1) determined in a method according to the present disclosure is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), (HexNAc(3)Hex(4)NeuAc(1), HexNAc(5)Hex(6)Fuc(1)NeuAc(1), HexNAc(5)Hex(6)Fuc(3), HexNAc(4)Hex(5)Fuc(3)NeuAc(2), HexNAc(4)Hex(5)Fuc(1)NeuAc(1), HexNAc(5)Hex(6)Fuc(1)NeuAc(3) and HexNAc(6)Hex(7)NeuAc(1). In embodiments, the glycan structure at position N184 of haptoglobin (i.e. of the β-chain of haptoglobin having the sequence given in SEQ ID NO: 1) determined in a method according to the present disclosure is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), In embodiments, the glycan structure at position N184 of haptoglobin (i.e. of the β-chain of haptoglobin having the sequence given in SEQ ID NO: 1) determined in a method according to the present disclosure is HexNAc(5)Hex(6)Fuc(1)NeuAc(2).

In embodiments, an increased amount of the determined glycan structure at position N184 of haptoglobin (i.e. of the β-chain of haptoglobin having the sequence given in SEQ ID NO: 1) vis-à-vis the reference amount may be indicative of HCC (e.g. early HCC).

In embodiments in which the glycan structure HexNAc(5)Hex(6)Fuc(1)NeuAc(2) at position N184 of haptoglobin is determined an increased amount of this glycan structure at position N184 of haptoglobin (i.e. of the β-chain of haptoglobin having the sequence given in SEQ ID NO: 1) vis-à-vis the reference amount may be indicative of HCC (e.g. early HCC).

In a particularly preferred embodiment the glycan structure at position N184 of haptoglobin (i.e. of the β-chain of haptoglobin having the sequence given in SEQ ID NO: 1) determined is HexNAc(5)Hex(6)Fuc(1)NeuAc(2).

In embodiments of the method according to the first aspect, the method further comprises determining the amount of PIVKA-II and/or the amount of AFP in the sample or another sample from the same subject and wherein the score for detecting HCC takes into account the determined amount of PIVKA-II and/or the determined amount of AFP.

As demonstrated in the appended examples combining the amount of a glycan structure at position N184 of haptoglobin with AFP and/or PIVKA-II increases diagnostic utility for HCC (e.g. early HCC).

In a second aspect, the present disclosure relates to an in vitro method for aiding in the detection of HCC (e.g. early HCC) in a subject comprising the steps of: a) determining the amount of the N-glycan structure HexNAc(5)Hex(6)Fuc(1)NeuAc(2) at position N184 of haptoglobin (i.e. of the β-chain of haptoglobin having the sequence given in SEQ ID NO: 1) in a sample obtained from said subject and b) (i) comparing the amount of said N-glycan structure determined in a) to a reference amount of said N-glycan structure, wherein an altered (e.g. increased) amount of HexNAc(5)Hex(6)Fuc(1)NeuAc(2) at position 184 of haptoglobin in said subject's sample relative to the reference amount of this N-glycan structure is indicative for HCC (e.g. early HCC); or (ii) determining a score for detecting HCC (e.g. early HCC) taking into account the amount HexNAc(5)Hex(6)Fuc(1)NeuAc(2) at position N184 of haptoglobin determined in a) and comparing the determined score for detecting HCC (e.g. early HCC) to a reference value for said score indicative for HCC (e.g. early HCC).

Based on the comparison in b) i) or ii) the presence or absence of HCC (e.g. early HCC) may be determined.

The method of the second aspect is based on the finding that an increased amount of HexNAc(5)Hex(6)Fuc(1)NeuAc(2) at position 184 of haptoglobin (i.e. of the β-chain of haptoglobin having the sequence given in SEQ ID NO: 1) has been found in samples obtained from subjects suffering from HCC (in particular early HCC) vis-à-vis control samples representing chronic liver diseases, incl. HBV, HCV and cirrhosis. As illustrated by the appended Examples, the N-glycan structure HexNAc(5)Hex(6)Fuc(1)NeuAc(2) at position N184 of haptoglobin (i.e. of the β-chain of haptoglobin having the sequence given in SEQ ID NO: 1) represent a very promising marker for HCC (e.g. early HCC), showing the highest AUC in detection of HCC (combining early and late HCC) of all N-glycan structures identified (including all glycan structures at position N184 but also the other glycosylation sites). The structure of HexNAc(5)Hex(6)Fuc(1)NeuAc(2) may be as shown in FIG. 2. Accordingly, in embodiments, the N-glycan structure HexNAc(5)Hex(6)Fuc(1)NeuAc(2) at position N184 of haptoglobin (i.e. of the β-chain of haptoglobin having the sequence given in SEQ ID NO: 1) may have the glycan structure as depicted in the glycan part of the glycopeptide in FIG. 2 or the glycan structure of formula 2 as referred to herein elsewhere.

What is said herein with respect to the method according to the first aspect of the invention applies mutatis mutandis.

In embodiments of the second aspect of the invention, provided is an in vitro method for aiding in the detection of HCC (e.g. early HCC) in a subject comprising the steps of: a) determining the amount of the N-glycan structure HexNAc(5)Hex(6)Fuc(1)NeuAc(2) at position N184 of haptoglobin (i.e. of the β-chain of haptoglobin having the sequence given in SEQ ID NO: 1) in a sample obtained from said subject and b) comparing the amount of said N-glycan structure determined in a) to a reference amount of said N-glycan structure, wherein an altered amount of HexNAc(5)Hex(6)Fuc(1)NeuAc(2) at position N184 of haptoglobin in said subject's sample relative to the reference amount of this N-glycan structure is indicative for HCC (e.g. early HCC).

If the reference amount of the N-glycan structure HexNAc(5)Hex(6)Fuc(1)NeuAc(2) is selected such that it is representative for the amount of said N-glycan structure in healthy subjects and/or subjects suffering from non-cancerous chronic liver diseases (e.g. selected from the group consisting of HBV, HCV and cirrhosis), an increased amount of HexNAc(5)Hex(6)Fuc(1)NeuAc(2) at position N184 of haptoglobin (i.e. of the β-chain of haptoglobin having the sequence given in SEQ ID NO: 1) relative to the reference amount is indicative of HCC (e.g. early HCC).

Accordingly, in embodiments, an increase in the N-glycan structure HexNAc(5)Hex(6)Fuc(1)NeuAc(2) is indicative for HCC (e.g. early HCC).

In embodiments, the second aspect of the invention relates to an in vitro method for aiding in the detection of HCC (e.g. early HCC) in a subject comprising the steps of: a) determining the amount of the N-glycan structure HexNAc(5)Hex(6)Fuc(1)NeuAc(2) at position N184 of haptoglobin (i.e. of the β-chain of haptoglobin having the sequence given in SEQ ID NO: 1) in a sample obtained from said subject and b) determining a score for detecting HCC (e.g. early HCC) taking into account the amount HexNAc(5)Hex(6)Fuc(1)NeuAc(2) at position N184 of haptoglobin determined in a); and c) comparing the determined score for detecting HCC (e.g. early HCC) to a reference value for said score indicative for HCC (e.g. early HCC). The comparison in c) may then be used to determine the presence or absence of HCC (e.g. early HCC).

In embodiments, the score may be configured such that the a higher value of the score indicates an increased risk for HCC. In these embodiments, the score value would increase with an increase in the level of the N-glycan structure HexNAc(5)Hex(6)Fuc(1)NeuAc(2) at position N184 of haptoglobin.

Determining the amount of the glycan structure HexNAc(5)Hex(6)Fuc(1)NeuAc(2) at position N184 of haptoglobin (i.e. of the β-chain of haptoglobin having the sequence given in SEQ ID NO: 1) in a sample means that a measure reflecting the absolute or relative amount of haptoglobin glycosylated with the respective glycan structure at position N184 of hapoglobin in the sample is determined.

Embodiments for determining the amount of a glycan structure, such as the glycan structure HexNAc(5)Hex(6)Fuc(1)NeuAc(2) at position N184 of haptoglobin (i.e. of the β-chain of haptoglobin having the sequence given in SEQ ID NO: 1), are disclosed herein below.

In embodiments, the level of the glycan structure HexNAc(5)Hex(6)Fuc(1)NeuAc(2) at position N184 of haptoglobin (i.e. of the β-chain of haptoglobin having the sequence given in SEQ ID NO: 1) may be determined by determining the level of a glycopeptide comprising N184 and the glycan structure, e.g. via mass spectrometry.

In embodiments, the glycopeptides having the glycan structure HexNAc(5)Hex(6)Fuc(1)NeuAc(2) at position N184 of haptoglobin (i.e. of the β-chain of haptoglobin having the sequence given in SEQ ID NO: 1) used for determining the amount of HexNAc(5)Hex(6)Fuc(1)NeuAc(2) at position N184 of haptoglobin (i.e. of the β-chain of haptoglobin having the sequence given in SEQ ID NO: 1) comprise the peptide sequence of MVSHHNLTTGATLINE (SEQ ID NO: 2), wherein the N in position 6 of SEQ ID NO: 2 corresponds to the N at position 184 of the β-chain of haptoglobin (SEQ ID NO: 1). In specific embodiments, the peptide part of the glycopeptides may consist of MVSHHNLTTGATLINE (SEQ ID NO: 2). Accordingly, the amount of the glycopeptide of formula 1 (see elsewhere herein) may be determined for determining the amount of the glycan HexNAc(5)Hex(6)Fuc(l)NeuAc(2) at position N184 of haptoglobin. Depending on the sample preparation and method used for determining the methionine in position 1 of SEQ ID NO: 2 may be oxidized when the level of the glycopeptide is measured.

In embodiments, the glycopeptide having the glycan structure HexNAc(5)Hex(6)Fuc(1)NeuAc(2) at position N184 of haptoglobin (i.e. of the β-chain of haptoglobin having the sequence given in SEQ ID NO: 1) detected for determining the amount of the N-glycan HexNAc(5)Hex(6)Fuc(1)NeuAc(2) at position N184 of haptoglobin has an m/z of 1118,2098, a charge of 4 and a glycopeptide mass of 4469,8163 Da.

In other words, for determining the amount of the glycan structure HexNAc(5)Hex(6)Fuc(1)NeuAc(2) at position N184 of haptoglobin (i.e. of the β-chain of haptoglobin having the sequence given in SEQ ID NO: 1) a glycopeptide may be analyzed, wherein said glycopeptide may comprise the peptide sequence MVSHHNLTTGATLINE (SEQ ID NO: 2), wherein the N in position 6 of SEQ ID NO: 2 corresponds to the N at position 184 of the β-chain of haptoglobin (SEQ ID NO: 1) and wherein the glycan structure is HexNAc(5)Hex(6)Fuc(1)NeuAc(2). In specific embodiments, the peptide part of the glycopeptide may consist of MVSHHNLTTGATLINE (SEQ ID NO: 2). Accordingly, for determining the amount of the glycan structure HexNAc(5)Hex(6)Fuc(1)NeuAc(2) at position N184 of haptoglobin a glycopeptide of formula 1 (see herein elsewhere) may be analyzed.

In embodiments, for determining the amount of the glycan structure HexNAc(5)Hex(6)Fuc(1)NeuAc(2) at position N184 of haptoglobin (i.e. of the β-chain of haptoglobin having the sequence given in SEQ ID NO: 1) a glycopeptide may be analyzed which has the amino acid sequence MVSHHNLTTGATLINE (SEQ ID NO: 2), wherein the N in position 6 of SEQ ID NO:2 corresponds to the N at position 184 of the β-chain of haptoglobin (SEQ ID NO: 1) and wherein the glycan is HexNAc(5)Hex(6)Fuc(1)NeuAc(2). This glycopeptide may be the glycopeptide as shown in Formula 1 (see herein elsewhere) or FIG. 2. It has a characteristic m/z-value and also is well-defined this alternative way. E.g. a glycopeptide for determining the amount of the glycan structure HexNAc(5)Hex(6)Fuc(1)NeuAc(2) at position N184 of haptoglobin may have an m/z of 1118,2098, a charge of 4 and a glycopeptide mass of 4469,8163 Da. In embodiments, the glycopeptide may comprise the amino acid sequence MVSHHNLTTGATLINE (SEQ ID NO: 2) as peptide part and HexNAc(5)Hex(6)Fuc(1)NeuAc(2) as glycan structure attached to the N in position 6 of SEQ ID NO:2, wherein the N in position 6 of SEQ ID NO:2 corresponds to the N at position 184 of the β-chain of haptoglobin (SEQ ID NO: 1) sequence, and may have an m/z of 1118,2098927.9544, a charge of 5 4 and a glycopeptide mass of 4469,81634636.8124 Da.

To reduce the impact of sample handling and differences in the absolute amounts of haptoglobin between individuals, determining the amount of the glycan structure HexNAc(5)Hex(6)Fuc(1)NeuAc(2) at position N184 of haptoglobin (i.e. of the β-chain of haptoglobin having the sequence given in SEQ ID NO: 1) may mean determining a relative amount with respect to the amount of a second analyte or a group of anayltes. Such second analyte may be a haptoglobin peptide or haptoglobin glycopeptide. The peptide or glycopeptide may be externally spiked into the sample before the determination step or may be a peptide or gylcopeptide of haptoglobin contained in the sample. Spiked peptides may be labeled with heavy isotopes.

In embodiments of the method according to the second aspect, the method further comprises determining the amount of PIVKA-II and/or the amount of AFP in the sample or another sample from the same subject and wherein the score for detecting HCC takes into account the determined amount of PIVKA-II and/or the determined amount of AFP. As demonstrated in the appended examples combining the amount of a glycan structure at position N184 of haptoglobin with at least AFP increases diagnostic utility for HCC (e.g. early HCC).

In a third aspect, the present invention relates to an isolated glycopeptide having a peptide part and a N-glycan part, wherein the peptide part comprises or consists of the amino acid sequence MVSHHNLTTGATLINE (SEQ ID NO: 2) and wherein the N-glycan part is HexNAc(5)Hex(6)Fuc(1)NeuAc(2), and wherein the N-glycan part is attached to the N in position 6 of SEQ ID NO: 2. In embodiments, the methionine in position 1 of SEQ ID NO: 2 may be oxidized.

In a preferred embodiment, the glycopeptide is a gylcopeptide of Formula 1:

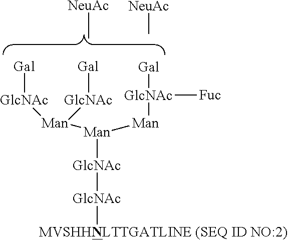

wherein GlcNAc means N-acetylglucosamine, Man means Mannose, Gal means Galactose, Fuc means Fucoseand NeuAc means N-Acetyl-neuraminic acid (sialic acid). Lines represent covalent bonds. The glycan in the glycopeptide is covalently attached as N-glycan via the indicated GlcNac of the glycan to the side chain of an asparagin corresponding to N184 of haptoglobin. (i.e. N184 of the β-chain of haptoglobin of SEQ ID NO: 1).

The isolated glycopeptide of Formula 1 is shown in a merely graphically alternative representation in FIG. 2 (right panel). The right panel of FIG. 2 is a representation according to the SNFG (Symbol Nomenclature for Glycans) system (Ajit Varki et al., Symbol Nomenclature for Graphical Representations of Glycans, Glycobiology, Volume 25, Issue 12, December 2015, Pages 1323-1324, https://doi.org/10.1093/glycob/cwv09l and Sriram Neelamegham et a.al, The SNFG Discussion Group, Updates to the Symbol Nomenclature for Glycans guidelines, Glycobiology, Volume 29, Issue 9, September 2019, Pages 620-624, https://doi.org/10.1093/glycob/cwz045)

As shown throughout the present disclosure the glycopeptide of Formula 1 holds great promise for the detection of HCC (e.g. early HCC). It was found herein to be a reliable biomarker for detecting early and late HCC.

Thus, in a fourth aspect, the present disclosure relates to the use of the glycopeptide according to the fifth aspect of the present disclosure (i.e. of Formula 1) in the detection of HCC (e.g. early HCC).

The embodiments of the methods according to the first and second aspect apply to that use mutatis mutandis.

According to a fifth aspect of the present disclosure, provided is a method for detecting a glycan structure at position N184 of haptoglobin (i.e. of the β-chain of haptoglobin having the sequence given in SEQ ID NO: 1), the method comprising the steps of: a) purifying haptoglobin from a sample to be analyzed; b) digesting the haptoglobin obtained in step a) by GluC and trypsin, and c) detecting the glycopeptide comprising position N184 of haptoglobin with the glycan structure attached thereto generated in b), thereby detecting the glycan structures at position N184.

In embodiments, the glycan structure at position N184 of haptoglobin (i.e. of the β-chain of haptoglobin having the sequence given in SEQ ID NO: 1) is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), (HexNAc(3)Hex(4)NeuAc(1), HexNAc(5)Hex(6)Fuc(1)NeuAc(1), HexNAc(5)Hex(6)Fuc(3), HexNAc(4)Hex(5)Fuc(3)NeuAc(2), HexNAc(4)Hex(5)Fuc(1)NeuAc(1), HexNAc(5)Hex(6)Fuc(1)NeuAc(3) and HexNAc(6)Hex(7)NeuAc(1). In embodiments, the glycan structure at position N184 of haptoglobin (i.e. of the β-chain of haptoglobin having the sequence given in SEQ ID NO: 1) is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), In particularly preferred embodiments, the glycan structure at position N184 of haptoglobin is HexNAc(5)Hex(6)Fuc(1)NeuAc(2).

In the appended Examples, it is shown that the glycopeptides comprising an N-glycan modification at the asparagine corresponding to position N184 of haptoglobin can be reliably determined with good sensitivity after purification and digestion and performing an appropriate detection method, preferably LC-MS. In particular, all glycopeptides specifically referred to herein (e.g. the glycopeptide according to the fifth aspect of the invention) could be reliably determined using such method.

The appended Examples in particular also show that a glycopeptide according to the forth aspect of the present disclosure can be reliably determined with good sensitivity after purification and digestion and performing an appropriate detection method, preferably LC-MS. Accordingly, in embodiments, the provided is a method for detecting the glycan structure of the glycopeptide according to the forth aspect (e.g. the glycopeptide of Formula 1), the method comprising the steps of: a) purifying haptoglobin from a sample to be analyzed; b) digesting the haptoglobin obtained in step a) by GluC and trypsin, and c) detecting the glycopeptides obtained in b), thereby detecting the glycopeptide according to the forth aspect (e.g. the glycopeptide of Formula 1).

In the clinical routine the diagnosis of HCC, especially the diagnosis of HCC at an early stage, is quite challenging and many cases of HCC are diagnosed too late and cannot be cured any more. Ultra-sound is used frequently to “look” for the presence of liver cancer. However, the results obtained by ultrasound vary from clinician to clinician, depend on the quality of the ultra-sound appliance used and are impacted by factors like body mass/fat. Nonetheless, later stages of HCC are often first spotted via ultra-sound.

It is envisaged that the glycan structures disclosed in the present invention will significantly improve the detection of HCC, especially the detection of early stage HCC. In one embodiment a method of the present disclosure for detection of HCC is a method for the detection of HCC at an early stage (i.e. early HCC).

The glycan structure of the present invention may also be combined with other biomarkers for the detection of HCC (e.g. AFP).

In a sixth aspect, the present disclosure relates to an (in vitro) method for aiding in the detection of HCC (e.g. early HCC) in a subject comprising the steps of: a) determining the amount of a N-glycan structure at position N184 of haptoglobin (i.e. of the β-chain of haptoglobin having the sequence given in SEQ ID NO: 1) in a sample obtained from said subject, b) determining the amount of a HCC biomarker other than the N-glycan structure at position N184 in a) in a sample obtained from said subject, c) combining the amounts determined in a) and b) to a combined value (e.g. a score for detecting HCC, e.g. early HCC) and comparing the combined value to a reference value for said combined value, wherein an altered combined value is indicative for HCC (e.g. early HCC).

The embodiments of the foregoing aspects apply mutatis mutandis.

The HCC biomarker is a biomarker indicative for HCC (e.g. early HCC). In specific examples, the HCC biomarker may be AFP or PIVKA-II.

In embodiments of the sixth aspect of the invention, provided is an (in vitro) method for aiding in the detection of HCC (e.g. early HCC) in a subject comprising the steps of: a) determining the amount of a N-glycan structure at position N184 of haptoglobin (i.e. of the β-chain of haptoglobin having the sequence given in SEQ ID NO: 1) in a sample obtained from said subject, b) determining the amount of AFP and/or the amount of PIVKA-II in a sample obtained from said subject, c) combining the amounts determined in a) and b) to a combined value (e.g. a score for detecting HCC, e.g. early HCC) and comparing the combined value to a reference value for said combined value, wherein an altered combined value is indicative for HCC (e.g. early HCC).

In embodiments of the method of the sixth aspect, provided is an (in vitro) method for aiding in the detection of HCC (e.g. early HCC) in a subject comprising the steps of: a) determining the amount of a N-glycan structure at position N184 of haptoglobin (i.e. of the β-chain of haptoglobin having the sequence given in SEQ ID NO: 1) in a sample obtained from said subject, b) determining the amount of AFP in a sample obtained from said subject, c) combining the amounts determined in a) and b) to a combined value (e.g. a score for detecting HCC, e.g. early HCC) and comparing the combined value to a reference value for said combined value, wherein an altered combined value is indicative for HCC (e.g. early HCC).

In embodiments of the method of the sixth aspect, provided is an (in vitro) method for aiding in the detection of HCC (e.g. early HCC) in a subject comprising the steps of: a) determining the amount of a N-glycan structure at position N184 of haptoglobin (i.e. of the β-chain of haptoglobin having the sequence given in SEQ ID NO: 1) in a sample obtained from said subject, b) determining the amount of PIVKA-II in a sample obtained from said subject, c) combining the amounts determined in a) and b) to a combined value (e.g. a score for detecting HCC, e.g. early HCC) and comparing the combined value to a reference value for said combined value, wherein an altered combined value is indicative for HCC (e.g. early HCC).

In embodiments of the method of the sixth aspect, provided is an (in vitro) method for aiding in the detection of HCC (e.g. early HCC) in a subject comprising the steps of: a) determining the amount of a N-glycan structure at position N184 of haptoglobin (i.e. of the β-chain of haptoglobin having the sequence given in SEQ ID NO: 1) in a sample obtained from said subject, b) determining the amount of PIVKA-II and the amount of AFP in a sample obtained from said subject, c) combining the amounts determined in a) and b) to a combined value (e.g. a score for detecting HCC, e.g. early HCC) and comparing the combined value to a reference value for said combined value, wherein an altered combined value is indicative for HCC (e.g. early HCC).

In embodiments, the glycan structure at position N184 of haptoglobin (i.e. of the β-chain of haptoglobin having the sequence given in SEQ ID NO: 1) is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), HexNAc(3)Hex(4)NeuAc(1), HexNAc(5)Hex(6)Fuc(1)NeuAc(1), HexNAc(5)Hex(6)Fuc(3), HexNAc(4)Hex(5)Fuc(3)NeuAc(2), HexNAc(4)Hex(5)Fuc(1)NeuAc(1), HexNAc(5)Hex(6)Fuc(1)NeuAc(3) and HexNAc(6)Hex(7)NeuAc(1). In embodiments, the glycan structure at position N184 of haptoglobin (i.e. of the β-chain of haptoglobin having the sequence given in SEQ ID NO: 1) is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), In particularly preferred embodiments, the glycan structure at position N184 of haptoglobin is HexNAc(5)Hex(6)Fuc(1)NeuAc(2).

What has been said with respect to the determination of the amount of the respective glycan structures herein elsewhere (e.g. in the context of other aspects and herein below) applies mutatis mutandis. Accordingly, the amount of the glycan structures may be determined be determining the amount of glycopeptides (e.g. via mass spectrometry).

The sample used for the determination(s) in a) and b) may be the same or may be different samples obtained from the subject. Preferably, the samples have been obtained from the subject at the same time. In a specific embodiment the sample for the determination(s) in a) is the same as for the determination(s) in b).

“The combined value” may in embodiments mean determining a score indicative for HCC (e.g. early HCC), said score taking into account the amounts determined in a) and b). The score may further take into account further biomarkers. The score may alternatively and additionally further take into account clinical data of the subject (e.g. age, gender, smoking status, cancer history, family cancer history and the presence of pre-existing liver diseases).

Exemplary but non-limiting ways for calculating a combined value or score are disclosed herein below.

Methods for determining the amount of AFP and PIVKA-II are well known in the art. For instance, microchip capillary electrophoresis and a liquid-phase binding assay on the uTASWako i30 automated analyzer (Fujifilm Wako Pure Chemical Industries, Osaka, Japan), according to the instructions of the manufacturer. AFP may, for example, also be detected with Elecsys® AFP (Material number: 044817981190). PIVKA-II may, for example, be detected using Elecsys® PIVKA-II (Material number: 08333602190).

In the method according to the sixth aspect, the N-glycan structure is preferably HexNAc(5)Hex(6)Fuc(1)NeuAc(2), even more preferably the glycan as comprised in the glycopeptide of formula 1 (see above). In further embodiments of these aspects and embodiments, also the amount of HexNAc(4)Hex(5)NeuAc(2) at position N184 or HexNAc(5)Hex(5)NeuAc(1) at position N207 of haptoglobin (i.e. of the β-chain of haptoglobin having the sequence given in SEQ ID NO: 1) may be determined; a ratio of the amounts may be calculated and the combined value may be calculated using such ratio.

In a seventh aspect, provided herein is a computer-implemented method for aiding in the detection of HCC (e.g. early HCC) in a subject, said method comprising:
a) receiving data comprising the amount of a glycan structure at position N184 of haptoglobin (i.e. of the β-chain of haptoglobin having the sequence given in SEQ ID NO: 1) in a sample obtained from said subject; and
b) i) comparing the amount of the glycan structure received in a) to a reference amount of said N-glycan structure at position N184 of haptoglobin, wherein an altered amount of the N-glycan structure in said patient sample relative to the reference amount of said one or more glycan structure is indicative for HCC; or (ii) calculating a score for detecting HCC (e.g. early HCC) taking into account the amount of said glycan structure at position N184 of haptoglobin received in a) and comparing the calculated score for detecting HCC (e.g. early HCC) to a reference value for said score indicative for HCC (e.g. early HCC).

The embodiments of the foregoing aspects apply mutatis mutandis.

In embodiments, the method may comprise aiding in detection of HCC (e.g. early HCC) based on i) or ii) in b).

In embodiments, the method may comprise outputting (e.g. via a display) whether the subject suffers from HCC (e.g. early HCC) or not.

In embodiments, the glycan structure at position N184 of haptoglobin (i.e. of the β-chain of haptoglobin having the sequence given in SEQ ID NO: 1) is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), HexNAc(3)Hex(4)NeuAc(1), HexNAc(5)Hex(6)Fuc(1)NeuAc(1), HexNAc(5)Hex(6)Fuc(3), HexNAc(4)Hex(5)Fuc(3)NeuAc(2), HexNAc(4)Hex(5)Fuc(1)NeuAc(1), HexNAc(5)Hex(6)Fuc(1)NeuAc(3) and HexNAc(6)Hex(7)NeuAc(1). In embodiments, the glycan structure at position N184 of haptoglobin (i.e. of the β-chain of haptoglobin having the sequence given in SEQ ID NO: 1) is selected from the group consisting of HexNAc(5)Hex(6)Fuc(1)NeuAc(2); HexNAc(2)Hex(8), HexNAc(2)Hex(9), HexNAc(5)Hex(6)Fuc(3)NeuAc(1), HexNAc(4)Hex(5)Fuc(3), In particularly preferred embodiments, the glycan structure at position N184 of haptoglobin is HexNAc(5)Hex(6)Fuc(1)NeuAc(2).

In an eighth aspect, provided is a computer-implemented method for aiding in the detection of hepatocellular carcinoma (HCC) in a subject comprising the steps of:
a) receiving data comprising the amount of the glycan structure HexNAc(5)Hex(6)Fuc(1)NeuAc(2) at position N184 of haptoglobin (i.e. of the β-chain of haptoglobin having the sequence given in SEQ ID NO: 1) in a sample obtained from said subject and
b) i) comparing the amount of HexNAc(5)Hex(6)Fuc(1)NeuAc(2) received in a) to a reference amount of said glycan structure, wherein an increased amount of HexNAc(5)Hex(6)Fuc(1)NeuAc(2) at position N184 of haptoglobin in said subject's sample relative to the reference amount of this glycan structure is indicative for HCC, or (ii) calculating a score for detecting HCC (e.g. early HCC) taking into account the amount of HexNAc(5)Hex(6)Fuc(1)NeuAc(2) at position N184 of haptoglobin received in a) and comparing the calculated score for detecting HCC (e.g. early HCC) to a reference value for said score indicative for HCC (e.g. early HCC).

The embodiments of the foregoing aspects apply mutatis mutandis.

In embodiments, the method may comprise aiding in detection of HCC (e.g. early HCC) based on i) or ii) in b).

In embodiments, the method may comprise outputting (e.g. via a display) whether the subject suffers from HCC (e.g. early HCC) or not.

If there are two or more steps of receiving data in the computer-implemented methods disclosed herein, a skilled person will readily understand that this includes both (i) embodiments in which one single data set including all the received data in the two or more steps and (ii) in which two or more separate data sets are received.

Any of the computer implemented methods mentioned above may optionally comprise the step of outputting whether the subject suffers or is suspected to suffer from HCC (e.g. early HCC) and/or whether further clinical checks for HCC (e.g. early HCC) are required based on the comparison step. The output may be via a display.

Also disclosed herein is a computer program product comprising instructions which, when the program is executed by a computer, cause the computer to carry out the computer-implemented method according to the seventh or eighth aspect.

Further, provided is a computer-readable medium comprising instructions which, when executed by a computer, cause the computer to carry out the computer-implemented method according to the seventh or eighth aspect.

Also provided is a data processing system comprising a receiving unit configured to receiving data as defined in the seventh or eighth aspect; and a processing unit configured to perform the calculation and/or comparison step and/or any determination/assessment step in the seventh or eighth aspect; and optionally an outputting unit configured to output the assessment results.

The above aspects relate to glycan structures at position N184 of the β-chain of haptoglobin. As evident from the appended examples, herein identified were also a number of other glycan structures at positions N207, N211 or N241 that are useful for detecting HCC.

The respective glycan structures at position N207 of haptoglobin are: HexNAc(6)Hex(7)Fuc(1)NeuAc(4); HexNAc(4)Hex(5)NeuAc(2); HexNAc(5)Hex(5)NeuAc(1); HexNAc(5)Hex(6)Fuc(1)NeuAc(1) HexNAc(5)Hex(6)Fuc(1)NeuAc(2); and HexNAc(6)Hex(7)Fuc(1)NeuAc(1).

Moreover, respective glycan structures at position N207 are provided in Tables 2 and 3.

The respective glycan structures at position N211 of haptoglobin are: HexNAc(6)Hex(7)NeuAc(1), HexNAc(5)Hex(6)Fuc(1)NeuAc(2), HexNAc(6)Hex(7)Fuc(1)NeuAc(1).

Moreover, respective glycan structures at position N211 are provided in Tables 2 and 3.

The respective glycan structures at position N241 of haptoglobin are: HexNAc(5)Hex(6)Fuc(1)NeuAc(1), HexNAc(4)Hex(5)NeuAc(2), HexNAc(5)Hex(6)Fuc(1)NeuAc(2), HexNAc(4)Hex(5)Fuc(1)NeuAc(1).

Moreover, respective glycan structures at position N241 are provided in Tables 2 and 3.

Accordingly, all aspects and embodiments as described herein for N184 of haptoglobin are disclosed for N207, N211 and N241 of haptoglobin mutatis mutandis. Further, all aspects and embodiments disclosed herein for specific glycan structures at position N184 are also disclosed for any of the above-mentioned glycan structures at position N207, N211 and N241 mutatis mutandis.

For detecting the amount of a glycan structure at position N207 a glycopeptide with a peptide part comprising or consisting of NLFLNHSE (SEQ ID NO: 3) may be used, wherein the N in position 5 corresponds to N207.

For detecting the amount of a glycan structure at position N211 a glycopeptide with a peptide part comprising or consisting of NATAK (SEQ ID NO: 4) may be used, wherein the N in position 1 corresponds to N211.

For detecting the amount of a glycan structure at position N241 a glycopeptide with a peptide part comprising or consisting of VVLHPNYSQVD (SEQ ID NO: 5) may be used, wherein the N in position 6 corresponds to N241.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The following definitions and embodiments apply to the present disclosure in its entirety, especially to all aspects and embodiments of the invention.

As used in this specification and the appended claims, the singular forms “a”, “an”, and “the” include plural referents, unless the content clearly dictates otherwise.

The word “comprise”, and variations such as “comprises” and “comprising”, will be understood to imply the inclusion of a stated integer or step or group of integers or steps but not the exclusion of any other integer or step or group of integers or steps.

The use of the alternative (e.g., “or”) should be understood to mean either one, both, or any combination thereof of the alternatives.

The term “and/or” should be understood to mean either one, or both of the alternatives.

As used herein and unless stated otherwise, it is to be understood that the term “about” is used synonymously with the term “approximately”. Illustratively and unless stated otherwise, the use of the term “about” when used in conjunction with a stated numerical value or range denotes somewhat more or somewhat less than the stated value or range, to within a range of ±15% of that stated, ±10% of that stated, ±5% of that stated, or conveniently ±2% of that stated. Such values are thus encompassed by the scope of the claims reciting the terms “about” or “approximately”.

The term “biomarker” or “marker” as used herein refers generally to a molecule, including a gene, protein, carbohydrate structure, or glycolipid, metabolite, mRNA, miRNA, protein, DNA (cDNA or genomic DNA), DNA copy number, or an epigenetic change, e.g., increased, decreased, or altered DNA methylation (e.g., cytosine methylation, or CpG methylation, non-CpG methylations); histone modification (e.g., (de)acetylation, (de) methylation, (de) phosphorylation, ubiquitination, SUMOylation, ADP-ribosylation); altered nucleosome positioning, the expression or presence of which in or on a mammalian tissue or cell can be detected by standard methods (or methods disclosed herein) and which may be predictive, diagnostic and/or prognostic for an individual's health or a disease. Therefore sometimes herein below the more general term “marker” is also used while discussing the more general terms and definitions. The term marker also includes a glycan structure or glycan, or a glycopeptide as analyzed in the present disclosure.

The term “in vitro method” is used to indicate that the method is performed outside a living organism and preferably on body fluids, isolated tissues, organs or cells. An in vitro method may also be referred to an ex vivo method.

Hepatocellular carcinoma (HCC) is the major histologic type among primary liver cancers occurring worldwide, accounting for 70% to 85% of the total burden. It is known, that underlying liver diseases such as liver fibrosis and cirrhosis are the main risk factors for the development of HCC. HCC can be treated by resection, liver transplantation, or local ablation with radiofrequency for patients diagnosed at an early stage. The 5-year survival rate of the HCC patients may be as high as 70% if this malignancy is diagnosed in an early stage. However, the 5-year survival rate of the HCC patients decreases significantly the later the disease is diagnosed and drops to only 15%, if HCC is diagnosed in the late stage of disease (Tsuchiya N, Sawada Y, Endo I, et al. Biomarkers for the early diagnosis of hepatocellular carcinoma. World J Gastroenterol. 2015; 21(37):10573-83; Siegel R, Naishadham D, Jemal A. Cancer statistics, 2013. CA: A Cancer Journal for Clinicians. 2013; 63(1):11-30).

Haptoglobin is an acute phase protein. It is synthesized in the liver and secreted into the plasma and has a rather complex biochemistry and diverse biological functions. As indicated in FIG. 1, haptoglobin can form dimers (or even polymers) via di-sulfide bonds. The basic form of haptoglobin is a dimer. The α-chain of human haptoglobin exhibits genetic polymorphism (α1-chain or α2-chain, respectively) resulting in the three types 1-1, 2-1 and 2-2 of haptoglobin (see FIG. 1). The concentration of haptoglobin in human plasma is usually in the range of 0.3-3 mg/ml. Haptoglobin binds to free hemoglobin and thereby prevents oxidative stress. It also plays a role in the regulation of immune response via binding to both resting and activated CD4+ and CD8+ T cells.

The β-chain of haptoglobin does not exhibit genetic polymorphism. As used herein the β-chain of haptoglobin preferably has the sequence as given in SEQ ID NO: 1. Haptoglobin can undergo secondary modifications, e.g. in form of glycosylation. The β-chain of haptoglobin has four N-glycosylation sites (asparagine (N)) at amino acid positions 183, 207, 211 and 241, respectively.

As used herein and if not otherwise mentioned, the expression “at position N184 of haptoglobin” refers to position N184 of the β-chain of haptoglobin (e.g. having the sequence given in SEQ ID NO: 1).

As will be understood by those skilled in the art being familiar with methods aiding in the detection of HCC (e.g. early HCC), the assessment of HCC made in accordance with the present invention, although preferred to be, may usually not be correct for 100% of the investigated subjects (as for essentially every diagnostic method). The term, typically, requires that a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley &amp; Sons, New York 1983. Typically envisaged confidence intervals are at least 50%, at least 60%, at least 70%, at least 80%, at least 90%, at least 95%. The p-values are, typically, 0.2, 0.1, 0.05.

The term “indicative for HCC” is used to illustrate that an increased level or amount of a marker (e.g. of a glycan structure or of the glycopeptide) determined and optionally its combination with other biomarkers or variables is very valuable but is not diagnostic without error but rather indicates with a high probability that the subject has HCC. Not in all (100%) of the patients with HCC the amount of the marker is above the reference level and not in all healthy individuals the level of the marker is lower than the reference level or cut-off level. As the skilled artisan will appreciate, in many diseases, no biochemical marker has 100% specificity and at the same time 100% sensitivity. Rather the marker analyzed or a marker combination comprising this marker gives a certain likelihood, e.g. at a given level of specificity or at a given level of sensitivity that an individual whose sample has been analyzed has a certain clinical status, e.g. has HCC. The skilled artisan is fully familiar with the mathematical/statistical methods used to calculate specificity, sensitivity, positive predictive value, negative predictive value, reference value or total error. Any of these parameters can be calculated and used to obtain an indication of the presence or absence of HCC.

The term “aiding in the detection of hepatocellular carcinoma (HCC)” is used to indicate that the method according to the present invention will help/aid a medical professional including, e.g., a physician in assessing whether an individual has HCC or is at risk of developing HCC. As will be appreciated several alternative methods (e.g. ultra sound, Radiography, MRT, or CT) can be used by a physician and combined with in vitro biomarker data, like glycan structure data, to detect or to exclude presence of HCC. The final diagnosis of HCC is usually made from a tissue biopsy or from a tissue sample after surgery. The term “aiding in the detection of HCC” includes that the method is used as sole diagnostic utility or is used as one of multiple diagnostic utilities.

In embodiments of the invention, “aiding in the detection of hepatocellular carcinoma HCC” may be “aiding in the detection of early HCC”. As demonstrated in the appended examples certain glycans at position 184 of the β-chain of haptoglobin show particularly good performance for detecting early HCC.

Unless defined otherwise, all technical and scientific terms used herein have the same meanings as commonly understood by one of ordinary skill in the art.

As used herein, the word “comprise”, and variations such as “comprises” and “comprising”, will be understood to imply the inclusion of a stated integer or step or group of integers or steps but not the exclusion of any other integer or step or group of integers or steps.

In aspects and embodiments set forth above certain marker values e.g. the amount of a glycan structure or the amount of a glycopeptide may be combined with the amount determined for one or more other biomarker (e.g. to a score). Such combination is performed using standard mathematical/statistical approaches.

One convenient goal to quantify the diagnostic accuracy of a laboratory test is to express its performance by a single number. The most common global measure is the area under the curve (AUC) of the ROC plot. The area under the ROC curve is a measure of the probability that the perceived measurement will allow correct identification of a condition (or the differentiation of one condition from the other). Values typically range between 1.0 (perfect separation of the test values of the two groups) and 0.5 (no apparent distributional difference between the two groups of test values). The area does not depend only on a particular portion of the plot such as e.g. the point closest to the diagonal or the sensitivity at 90% specificity, but on the entire plot. This is a quantitative, descriptive expression of how close the ROC plot is to the perfect one (area=1.0). In the context of the present invention, the two different conditions can be whether a patient has HCC or does not have HCC.

The present invention uses the terms “HCC”, “early HCC” and “late HCC”.

“Early HCC”, “early stage HCC” or “HCC of early stage” as used herein refers to patients classified to stages 0 and A according to Barcelona Clinic Liver Cancer (BCLC) classification (Llovet J M, Brd C, Bruix J, Semin Liver Dis. 1999; 19(3):329-38).

“Late HCC”, “late stage HCC” or “HCC of late stage” as used herein refers to patients classified to stages B, C and D according to Barcelona Clinic Liver Cancer (BCLC) classification (Llovet J M, Brd C, Bruix J, Semin Liver Dis. 1999; 19(3):329-38).

“HCC” refers to any form of HCC including early and late HCC.

BCLC classification is endorsed as the standard system for HCC management by the American Association for the Study of Liver Disease, American Gastroenterology Association, European Association for the Study of Liver and the European Organization for the Research and Treatment of Cancer. According to BCLC staging, patients are assigned to five categories (0, A, B, C and D): BCLC stage of 0 (defined as very early stage disease) comprises patients exhibiting a well-preserved liver function (Child-Pugh A; Cholongitas E, Papatheodoridis G V, Vangeli M, Terreni N, Patch D, Burroughs A K. Systematic review: The model for end-stage liver disease—should it replace Child-Pugh's classification for assessing prognosis in cirrhosis?. 2005; Alimentary Pharmacology & Therapeutics. 22 (11-12): 1079-89.) diagnosed with one asymptomatic nodule measuring less than 2 cm, without vascular invasion or satellites. A BCLC stage of A (defined as early-stage disease) includes patients with a Child-Pugh A or B status diagnosed with one nodule of any size or a maximum of three nodules measuring <3 cm. In context of the present disclosure BCLC stages 0 and A were defined as an Early HCC group. A BCLC stage of B (defined as intermediate-stage disease) corresponds to patients with a Child-Pugh grade A or B status diagnosed with multiple nodules without vascular invasion or extrahepatic metastasis. Patients with a Child-Pugh grade of A or B, vascular invasion or extrahepatic metastasis and cancer-related symptoms (PS 1-2) are classified as having BCLC C disease (defined as advanced-stage disease). Finally, patients with a Child-Pugh grade of C, in any tumor stage and cancer-related symptoms (PS >2) are classified as belonging to the BCLC D disease (defined as terminal stage disease). In the context of the present disclosure, patients of BCLC stages B, C and D were defined as a Late HCC group.

The term “subject” or “individual” as used herein relates to a single person. The subject may be healthy or a patient, e.g. having cirrhosis, being at risk of developing HCC, experiencing or having experienced one or more signs, symptoms, or other indicators of HCC. Intended to be included as a subject are any subjects involved in clinical research trials not showing any clinical sign of disease, or subjects involved in epidemiological studies, or subjects whose samples may serve as controls. In embodiments, the subject may be known to be at risk for developing HCC, e.g. through chronic alcohol consumption, hepatitis B and/or hepatitis C infection, non-alcoholic fatty liver disease, Wilson's disease, hereditary hemochromatosis, alphal-antitrypsin deficiency, primary biliary cirrhosis, autoimmune hepatitis and other risk factors.

In one embodiment according to the present disclosure, the subject from which the sample to be investigated had been obtained is a healthy subject and is screened for (the presence of) HCC as part of routine oncology surveillance.

In one embodiment according to the present disclosure, the subject from which the sample to be investigated had been obtained is a subject at risk for developing HCC and is screened for (the presence of) HCC as part of routine oncology surveillance.

A subject may be at risk to develop HCC if the subject is known to suffer from a chronic liver disease, viral- or non-viral hepatitis and/or liver cirrhosis.

In one embodiment according to the present disclosure the subject from which the sample to be investigated had been obtained has chronic liver disease, viral- or non-viral hepatitis, liver cirrhosis and is subjected to a differential diagnosis for presence or absence of HCC.

The term “sample”, “sample of a subject”, “patient sample” or an “individual's sample” as used herein refers to a biological sample obtained for the purpose of evaluation in vitro. In the methods of the present invention, the sample, patient sample or sample obtained from an individual preferably may be any type of body fluid. Body fluid sample includes blood, serum, plasma, urine, saliva, and synovial fluid. Preferred sample types are whole blood, serum or plasma. In one embodiment, the sample type is serum or plasma. In one embodiment, the sample type is plasma. In one embodiment, the sample type is serum. As the skilled artisan will appreciate, the sample is used for analysis of a marker of interest in vitro. The patient sample is discarded after the analysis. The patient sample is solely used for the in vitro method of the invention and the material of the patient sample is not transferred back into the patient's body.

The term “determining” the amount of an N-glycan structure or the amount of a glycopeptide, as used herein refers to the measuring of the amount or level of said N-glycan structure or of said glycopeptide. The level or the amount of an N-glycan structure or of a glycopeptide in the sample, is determined by employing any appropriate method, e.g. as known in the art or by employing a method as described herein.

In the context of the aspects of the present invention determining the amount of a glycan structure (e.g. HexNAc(5)Hex(6)Fuc(1)NeuAc(2) or any of the other glycan structures referred to herein) at position N184 of haptoglobin (i.e. of the β-chain of haptoglobin having the sequence given in SEQ ID NO: 1) in a sample means that a measure reflecting the absolute or relative amount of haptoglobin glycosylated with the respective glycan structure at position N184 of hapoglobin in the sample is determined. In embodiments, determining the amount of a glycan structure may be measuring the presence of said glycan structure and quantifying the amount/level of said glycan structure. In embodiments, the amount of a glycan structure at position N184 corresponds to the amount of a glycopeptide comprising said glycan structure at N184. Such glycopeptide may be generated by hydrolyzing the haptoglobin in the sample (e.g. using proteases, such as the proteases as described herein below).

In embodiments of the present disclosure, determining the amount of the glycan structure(s) (e.g. HexNAc(5)Hex(6)Fuc(1)NeuAc(2)) at position N184 of haptoglobin (i.e. of the β-chain of haptoglobin having the sequence given in SEQ ID NO: 1) may be determining a relative amount with respect to a second analyte (e.g. a control analyte). This setup allows to reduce the impact of sample handling and differences in the absolute amounts of haptoglobin between individuals. The second analyte may be a haptoglobin peptide or haptoglobin glycopeptide. The peptide may be spiked into the sample before the determination step or may be a peptide or glycopeptide of haptoglobin contained in the sample. Spiked peptides may be labeled with heavy isoptopes.

In the present disclosure determining the amount of a glycan structure at position N184 of haptoglobin (i.e. of the β-chain of haptoglobin having the sequence given in SEQ ID NO: 1) in a sample obtained from said subject may comprise generating glycopeptide(s) from the hatoglobin comprised in the sample, said glycopeptides comprising position N184 of haptoglobin (i.e. of the β-chain of haptoglobin having the sequence given in SEQ ID NO: 1) and (if present) a glycan structure attached thereto, and determining the amount(s) of the glycopeptide comprising the glycan of interest, e.g. via mass spectrometry. The amount of the respective glycan structure at position N184 of haptoglobin (i.e. of the β-chain of haptoglobin having the sequence given in SEQ ID NO: 1) may correspond or be derived from the amount of the respective glycopeptide comprising the respective glycan structure at position N184.

In an exemplary embodiment, determining the amount of a glycan structure at position N184 of haptoglobin (i.e. of the β-chain of haptoglobin having the sequence given in SEQ ID NO: 1) may comprise purifying haptoglobin (i.e. of the β-chain of haptoglobin having the sequence given in SEQ ID NO: 1) from the sample obtained from the subject, hydrolyzing the purified haptoglobin such that glycopeptides comprising position N184 of haptoglobin (i.e. of the β-chain of haptoglobin having the sequence given in SEQ ID NO: 1) and the respective glycans attached thereto are generated and determining the amount(s) of the glycopeptide having the glycan structure at position N184 of haptoglobin (i.e. of the β-chain of haptoglobin having the sequence given in SEQ ID NO: 1). The amount of respective glycan structure at position N184 of haptoglobin (i.e. of the β-chain of haptoglobin having the sequence given in SEQ ID NO: 1) may correspond or be derived from the amount of the respective glycopeptides comprising the respective glycan structure at position N184.

In the present disclosure determining the amount of a glycan structure at position N184 of haptoglobin (i.e. of the β-chain of haptoglobin having the sequence given in SEQ ID NO: 1) may, for example, comprise purification of haptoglobin from the sample, digestion of the purified haptoglobin with GluC and trypsin and MS-analysis determining the amount of the respective glycopeptide having the respective one or more glycan structures at position N184 of haptoglobin (i.e. of the β-chain of haptoglobin having the sequence given in SEQ ID NO: 1). The amount for the respective glycan structures at position N184 of haptoglobin (i.e. of the β-chain of haptoglobin having the sequence given in SEQ ID NO: 1) may correspond to or be derived from the determined amount of the corresponding glycopeptide comprising the respective glycan structure at position N184.

As the skilled artisan will appreciate, other methods can be used to determine the glycan structure at N184 of haptoglobin. It only has to be assured in such analysis that this glycan structure is attached or was attached to N184 of the haptoglobin β-chain. Non limiting examples for such methods are disclosed in Oh M J, Lee S H, Kim U, An HJ. Mass Spectrom Rev. 2021. Similar methods can be used for detecting glycan structures at positions N207, N211 and/or N241 of the haptoglobin β-chain.

In embodiments, the glycopeptide(s) comprising position N184 of haptoglobin used for determining the amount of a glycan structure may comprise the peptide sequence MVSHHNLTTGATLINE (SEQ ID NO: 2), wherein the N in position 6 of SEQ ID NO: 2 corresponds to the N at position 184 of the β-chain of haptoglobin (SEQ ID NO: 1) to which the respective glycan to be detected is attached. In specific embodiments, the peptide part of the glycopeptide(s) may consist of MVSHHNLTTGATLINE (SEQ ID NO: 2). In embodiments, the glycopeptide may be oxidized, in particular on the methionine. A skilled person will appreciate that such oxidation may occur depending on sample handling and methods used for detection of such glycopeptide.

In embodiments, a glycan structure for which the amount is determined is part of a glycopeptide, wherein said glycopeptide comprises the peptide sequence MVSHHNLTTGATLINE (SEQ ID NO: 2), and wherein N in position 6 of SEQ ID NO: 2 corresponds to the N at position 184 of the β-chain of haptoglobin (SEQ ID NO: 1).

In the context of the invention, the level or the amount of a N-glycan structure at position N184 of the β-chain of haptoglobin (e.g. HexNAc(5)Hex(6)Fuc(1)NeuAc(2) at position N184) or of a glycopeptide (e.g. of formula 1) in a sample, is determined. For this purpose any appropriate method known in the art may be employed. In embodiments of the methods according to the present disclosure, the step of determining one or more N-glycan structure at position N184 of the β-chain of haptoglobin (e.g. HexNAc(5)Hex(6)Fuc(1)NeuAc(2) at position N184) or of a corresponding glycopeptide (e.g. of formula 1) comprises that haptoglobin (comprising at least the β-chain) is purified, before such determination is performed. Preferably, the purification of haptoglobin is achieved via an antibody binding to haptoglobin. Especially a monoclonal antibody specifically binding to haptoglobin can be used. Such monoclonal antibody or an antigen-binding fragment thereof can be biotinylated and used in combination with streptavidin-coated magnetic beads (SA-beads). Non-limiting examples for such antibodies are commercially available and monoclonal antibodies detecting human Haptoglobin have been applied in different immunological methods in diverse publications. Non limiting examples are: Abcam, #AB13429, clone HG-36; Abnova, #MAB12976, clone 2F4; Acris Antibodies, #UM500010, clone UMAB10; Novus Biologicals, #NBP2-03008, clone OTI4H5; OriGene, #TA00399, clone OTI2B8; Thermo Fisher Scientific, #HYB 170-06-02, clone 9G10. In specific embodiments of the methods according to the present disclosure, the step of determining one or more N-glycan structure at position N184 of the β-chain of haptoglobin (e.g. HexNAc(5)Hex(6)Fuc(1)NeuAc(2) at position N184) or of a corresponding glycopeptide (e.g. of formula 1) comprises purifying haptoglobin (comprising at least the β-chain) via a biotinylated anti-haptoglobin monoclonal antibody in combination with streptavidin coated beads (SA-beads). In such purification step, the sample may be incubated with the biotinylated antibody and (simultaneously or sequentially) with (e.g. magnetic or magnetizable) SA-beads under conditions appropriate for antigen/antibody as well as biotin/streptavidin binding. The beads (having attached thereto the haptoglobin) are then separated from the other components comprised in the sample (e.g. by use of a magnetic force).

In embodiments of the present disclosure, the step of determining the amount of an (or one or more) N-glycan structure at position N184 of haptoglobin (e.g. HexNAc(5)Hex(6)Fuc(1)NeuAc(2) at position N184) comprises that haptoglobin is hydrolyzed chemically or biochemically/enzymatically cleaved/hydrolyzed into peptides/glycopeptides. Various methods for hydrolysis of polypeptides by chemicals are known and can be used. In embodiments, haptoglobin is enzymatically cleaved into peptides/glycopeptides.

In embodiments of the present disclosure, the step of determining the amount of an (or one or more) N-glycan structure at position N184 of haptoglobin (e.g. HexNAc(5)Hex(6)Fuc(1)NeuAc(2) at position N184) comprises that haptoglobin is enzymatically cleaved into peptides/glycopeptides using the enzymes GluC and trypsin. GluC cleaves protein chains C-terminal to the amino acid glutamic acid, whereas trypsin cleaves protein chains C-terminal to the amino acids arginine and lysine. Both enzymes cleave with high specificity.

As indicated above an (or one or more) N-glycan at position 184 of haptoglobin (e.g. HexNAc(5)Hex(6)Fuc(1)NeuAc(2) at position N184) or a glycopeptide comprising N184 (e.g. the glycopeptide of formula 1) can be measured by mass spectrometry (MS). Various MS-methods are known. Discovery and quantification of glycopeptides can be done by high resolution data dependent mass spectrometry (Mol Cell Proteomics. 2014 January; 13(1): 329-338). The mass spectrometer selects the most intense peptides entering the mass spectrometer at a given time and then selects the most intense species sequentially for gas-phase fragmentation with subsequent analysis of the obtained fragments. The fragments carry sequence information for the peptide, as well as for the glycan structure and are analyzed computer-assisted by comparing obtained fragment spectra with predicted spectra (derived from databases). In rare cases, manual inspection of the isotopic patterns is needed to differentiate between similar structures.

High sensitivity determination of glycopeptides can also be done by triple quadrupole mass spectrometry in single reaction monitoring mode (SRM)(Mol Cell Proteomics. 2013 April; 12(4): 1005-1016). In this technique, the mass spectrometer selects only the target peptide. This peptide is fragmented in the gas phase and a unique and representative fragment is determined and quantified.

For an MS-analysis of a glycan structure or of a glycopeptide quite a few handling steps are needed. These handling steps may cause quite some variation, especially regarding the measurement of a (absolute) level or amount. In order to compensate for differences in sample handling a sample is usually spiked with an internal standard (usually the analyte of interest but in an isotopically labelled form). Sample handling impacts the absolute values but is unlikely to impact a ratio of (glycan structures/glycopeptides) being based on the same method of MS-analysis. The ratio is not an absolute but rather a relative measure and thus is independent of differences in sample handling. Thus, it may be advantageous to determine amount of a glycan structure or a glycopeptide relative to another glycan structure or glycopeptide, respectively. In embodiments, the ratio may be between increased and decreased glycan structures at the same position of a polypeptide. Specific but non-limiting examples for such ratios are described herein elsewhere.

The term “amount” or “level” of an analyte (e.g. a glycan or glycopeptide) in a sample as used herein relates to any absolute measure which corresponds to the amount or concentration of said analyte in the sample or is proportional to the absolute amount or concentration of said analyte in the sample; or any relative measure, i.e. a measure representing an amount or concentration of the anaylte being relative to a reference amount or concentration, respectively. The reference amount or concentration may be the amount of an internal control (e.g. a standard glycopeptide, another glycopeptide detected in the sample or a peptide) measured simultaneously with the analyte of interest using the same methodology.

In embodiments, the amount of a glycan structure at position N184 of haptoglobin in a sample may be the amount of a glycopeptide comprising at N184 the glycan structure determined from the sample. In these embodiments the method may comprise generating glycopeptides from haptoglobin (e.g. via protease digest as described elsewhere). In embodiments, the peptide part of the glycopeptide may be as described above.

In embodiments, the amount of a glycopeptide may be the amount or raw signal for the glycopeptide divided by the amount or raw signal of a non-glycosylated peptide (e.g. from haptoglobin), respectively. In embodiments, the amount of a glycopeptide may be the amount or raw signal for the glycopeptide divided by the amount or raw signal of all detected glycopeptides (e.g. of haptoglobin), respectively

The term “altered amount” refers to the fact that an amount is outside the reference range, i.e. either above a certain reference amount or below a certain reference amount.

The terms “elevated” or “increased” level or amount of a marker refers to the amount or level of such marker in the sample investigated being higher in comparison to the amount or level (i.e. not including it) of such marker in a reference or control sample.

The term “decreased” level or amount of a marker refers to the amount or level of such marker in the sample investigated being lower (i.e. not including it) than the amount or level of such marker in a reference or control sample.

As used herein, the term “score” or “score for detecting HCC” taking into account the amount of a glycan structure or glycopeptide relates to a score (e.g. a value) obtained by combining the amount of a glycan structure or glycopeptide of the invention with at least one further parameter, such as, for example, an amount of a second glycan structure, an amount of one or more other biomarkers or clinical parameter. Exemplary but non-limiting examples for other biomarkers are AFP, PIVKA-II and AFP-L3. Exemplary examples for clinical parameters are age, gender, smoking status, ultrasound data, liver disease history, cancer history and family cancer history etc. The score is to be configured such that it can be indicative for HCC (e.g. early HCC).

“Taking into account” as used in the context of a score includes embodiments in which only the specifically listed amount(s) and parameters are taken into account and embodiments in which other non-listed parameters are taken into account.

Determining or calculating a score (e.g. a score for detecting HCC) can be achieved in many different ways. As referred to herein, combining certain biomarker data or other information to a combined value can be also referred to as determining or calculating a score.

Determining or calculating a score includes any mathematical combination of the amount of a glycan structure or glycopeptide as referred to herein with a further parameter (see above). Thus, a score may be calculated in that the individual parameters (including or consisting of the determined or received amounts of of the glycans attached to N184 of haptoglobin or respective glycopeptides) are mathematically combined. The levels may be used as such or may be mathematically transformed (e.g. by log transformation such as log 2 or log 10 transformation) for determining the score. The score may take into account one or more other factors than levels of PSA-glycoform species including but not limited to the presence or level of one or more other biomarkers in the sample and/or one or more clinical parameters of the subject (e.g. tumor histology, smoking status, stage of disease, and/or age).

In certain embodiments of the invention, a score for the detection of HCC may be obtained by or may comprise weighted calculation. This means that the biomarkers (e.g. the amounts of glycan structures at position N184 of haptoglobin) may be given different weightings. For example, when the score takes into account the amount of a first glycan structure at position N184 of haptoglobin and the amount of another glycan structure at position N184 of haptoglobin in the sample, the score may be calculated by the following equation:

Score=a*[amount first glycan structure]+b*[amount second glycan structure], wherein a and b represent the weighting factors. Preferably, the weighting factors or coefficients (in the above example a and b have been obtained by analyzing control samples such as from a reference population (e.g. any reference population as defined in the context of the reference value below). In embodiments, the weighting factors or coefficients may be obtained by a machine learning approach applied on a training data set obtained from samples of a reference population as defined herein.

A skilled person in the art is aware that a score and a corresponding reference value for the score can be optimized based on a reference population (e.g. any as defined herein or as disclosed in the appended Examples).

In embodiments, a score may be a binary score and the corresponding reference value may be binary. “Binary” means that the score contains two values, e.g. a first value being the level of the one or more di-antennary PSA-glycoforms determined or received or a value derived therefrom and a second value being the level of the one or more mono-antennary PSA-glycoforms or a value derived therefrom. A “value derived therefrom” can, for example, be a value obtained by a mathematical operation. The “value derived therefrom” is preferably directly proportional to the respective level. The values of the binary reference value may be obtained as described below for individual PSA-glycoforms.

Comparing a binary score to a reference value for a binary cut-off score means comparing the first value of the determined binary score to the first value of the reference value of the binary score and the second value of the determined binary score to the second value of reference value of the binary score. If a binary score is built from the amount of a first glycan structure at position N184 of haptoglobin and the amount of glycan structure at position N184 of haptoglobin, HCC may only be detected in the event that both amounts are indicatve for HCC relative to their reference values forming part of the binary score.

In the aspects and embodiments using two or more markers (or even more general input parameters which may also include clinical parameters such as age or gender) may be combined by an appropriate algorithm (e.g. logistic regression) derived from a multivariate analysis of the available data, preferably in an exhaustive search for feature selection. In such combination/calculation, a multivariate score is calculated. Other methods, e.g. selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods) can also be used to combine biomarker/input values into a score.

The performance of the results of the applied mathematical/statistical methods used in accordance with the present disclosure can be best described by their receiver operating characteristics (ROC). The ROC curve addresses both the sensitivity and the specificity of the test. Therefore, sensitivity and specificity values for a given biomarker or a combination of biomarkers are an indication of the performance of the test. For example, if a biomarker combination has a sensitivity value of 80%, 80 out of 100 diseased patients will be correctly identified, or if it has a specificity value of 80%, 80 out of 100 patients who do not have the disease will accurately test negative for the disease.

In the same manner as described before, the ROC curve can be used to access the performance of the discrimination between patients and controls by the aforementioned logistic regression model.

The present disclosure, in particular in context of the methods disclosed herein, refers to comparing the amount or level of a glycan structure, glycopeptide or score to a reference amount/level or score for said glycan structure, glycopeptide or score. It is to be understood that such comparison as used herein usually refers to a comparison of corresponding parameters, amounts, values or scores, e.g., an absolute amount is compared to an absolute reference amount while a concentration is compared to a reference concentration or an intensity signal obtained for the glycan structure in a sample is compared to the same type of intensity signal obtained from a reference sample. A score is typically compared to a reference value of such specific score being indicative for HCC (e.g. early HCC). The comparison may be carried out by an appropriate device, e.g. by a computer. The value of the measured or detected level/amount of the glycan structure in the sample from the individual or patient and the reference level/amount can be, e.g., compared to each other and the said comparison can be automatically carried out by a computer program executing an algorithm for the comparison. The computer program carrying out the said evaluation will provide the desired assessment in a suitable output format. For a computer assisted comparison, the value of the determined amount may be compared to values corresponding to suitable references, which are stored in a database by a computer program. The computer program may further evaluate the result of the comparison, i.e. automatically provide the desired assessment in a suitable output format. For a computer assisted comparison, the value of the determined amount may be compared to values corresponding to suitable references, which are stored in a database by a computer program. The computer program may further evaluate the result of the comparison, i.e. automatically provides the desired assessment in a suitable output format.

The term “glycan structure” or “glycan” are used interchangeably. In the present disclosure the glycans investigated are N-glycans, therefore the terms glycan and N-glycan herein are used interchangeably. A glycan or glycan structure consists of various types of carbohydrates. The glycan structure can e.g. be bound to an amino acid, for example to the amino acid asparagine. In the present case the glycans bound to the asparagine at position 184 of the β-chain of haptoglobin (SEQ ID NO: 1) are analyzed. The glycan structure or glycan of the present disclosure serves as a biomarker or marker of HCC (e.g. early HCC).

The term “glycopeptide” is used to refer to a peptide or to a peptide fragment of a larger polypeptide comprising an amino acid to which a glycan is covalently attached. In the present disclosure the glycopeptide preferably analyzed is a peptide sequence derived from the β-chain of haptoglobin, comprising the amino acid asparagine (N) at position 184 of the β-chain of haptoglobin (SEQ ID NO: 1). Preferred embodiments for the peptide part of the glycopeptide preferably detected or quantified are disclosed herein elsewhere.

The glycans are typically referred to herein with a sum formula. In the text we refer to a glycan composition as follows: HexNAc(x)Hex(x)Fuc(x)NeuAc(x). The numbers in the parentheses represent the number of monomers in the glycans. This nomenclature is well known to a skilled person working in the field of glycan biology. HexNAc can be GlcNAc or GalNAc (in embodiments GlcNAc) and Hex can be Glu or Gal (in embodiments Gal). HexNAc means N-acetylhexosamine, GlcNAc means N-acetylglucosamine, GalNAc means N-acetylgalactosamine, Hex means Hexose, Man means Mannose, Glu means Glucose, Gal means Galactose, Fuc means Fucose and NeuAc or Neu5Ac means N-Acetyl-neuraminic acid (sialic acid).

Preferred glycans are indicated with specific formulas or using schematic drawings according to the SNFG (Symbol Nomenclature for Glycans) system (Ajit Varki et al., Symbol Nomenclature for Graphical Representations of Glycans, Glycobiology, Volume 25, Issue 12, December 2015, Pages 1323-1324, https://doi.org/10.1093/glycob/cwv09l and Sriram Neelamegham et a.al, The SNFG Discussion Group, Updates to the Symbol Nomenclature for Glycans guidelines, Glycobiology, Volume 29, Issue 9, September 2019, Pages 620-624). An exemplary glycan is the glycan of formula 2.

In embodiments of the invention, the glycan HexNAc(5)Hex(6)Fuc(1)NeuAc(2) may have formula 2:

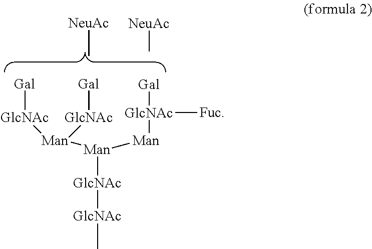

As used in the formulas (e.g. formula 1 and 2) herein GlcNAc means N-acetylglucosamine, Man means Mannose, Gal means Galactose, Fuc means Fucose and NeuAc means N-Acetyl-neuraminic acid (or sialic acid). Lines represent covalent bonds between the monosachharides. The lower GlcNac is the monosaccharide via which the glycan is attached to a peptide or protein (e.g. N184 of SEQ ID NO: 1) if is part of a glycopeptide or glycoprotein, respectively. The bracket used in context of NeuAc (or Neu5Ac which is used as synonym) indicate that the NeuAc can be attached to either of the three Galactose residues.

As used herein the term “reference amount” (or “reference level”) for an anylyte (e.g. of a glycan structure or a glycopeptide) refers to an independently established, predetermined amount of said analyte. As the skilled artisan will appreciate the reference amount is predetermined and set to meet routine requirements in terms of e.g. specificity and/or sensitivity for the purpose of detecting HCC (e.g. early HCC). Accordingly, the reference amount may be selected such that it is indicative for HCC (e.g. early HCC). The requirements for detecting HCC can vary, e.g. from regulatory body to regulatory body. It may for example be that assay sensitivity or specificity, respectively, has to be set to certain limits, e.g. to 80%, 90%, 95% or 98%, respectively. These requirements may also be defined in terms of positive or negative predictive values. For any selected requirement e.g. in terms of level of sensitivity or specificity, respectively, the reference range (if evaluated and decreased values are indicative of an abnormal status) or the reference level or cut-off level (if either evaluated or decreased values are indicative of an abnormal status) can be determined by the skilled artisan. In analogy and according to the same principles a reference ratio can be determined.

As used herein the term “reference value”, e.g. in the context of a reference value for a score, relates to an independently established, predetermined value for the respective parameter (e.g. a score). As the skilled artisan will appreciate the reference value is predetermined and set to meet routine requirements in terms of e.g. specificity and/or sensitivity for the purpose of detecting HCC (e.g. early HCC). Accordingly, the reference value may be selected such that it is indicative for HCC (e.g. early HCC). What is said above with respect to reference amount applies mutatis mutandis.

The reference amount or the reference value for a score is typically determined in a reference sample or in a reasonable number of reference samples. Reference samples are also called control samples. In general, a reference sample is obtained from an individual or a group of individuals known to suffer from, or known to be at risk of, a given condition; or from an individual or a group of individuals known to be free of a given condition, i.e., “normal” or “healthy” individual(s). Usually the sample's marker level is directly or indirectly correlated with a diagnosis and the marker level is e.g. used to determine whether an individual is at risk for HCC. Depending on the intended diagnostic use an appropriate reference sample is chosen and a control or reference value for the marker established therein. It will be appreciated by the skilled artisan that such reference sample in one embodiment is obtained from a reference population that is age-matched and free of confounding diseases. As also clear to the skilled artisan, the absolute marker values established in a reference sample or a set of reference samples (e.g form a reference population) will be dependent on the assay used. Preferably, samples from 100 well-characterized individuals from the appropriate reference population are used to establish a reference amount or reference value. Also preferred the reference population may be chosen to consist of 20, 30, 50, 200, 500 or 1000 individuals. Healthy individuals represent a frequently used reference population for establishing a control or reference amount or value. In one embodiment, the reference level or value is determined in reference samples from healthy individuals.

For detecting of HCC in the clinical routine, it is most critical to obtain an indication that HCC might develop or be present in those patients that have liver cirrhosis or other HCC risk factors. Accordingly, in embodiments the reference level is determined in reference samples from patients with liver cirrhosis.

In embodiments a reference population from which the control or reference samples may be obtained comprises samples obtained from control subjects (e.g. healthy individuals or subject with a liver disease with increased risk of developing HCC) and subjects suffering from HCC (e.g. early HCC).

As will be appreciated by a skilled person any method or use for detecting HCC or aiding in the detection of HCC may include the step of detecting HCC or aiding in the detection of HCC. This aid in detection or detection is typically achieved based on the comparison step of such methods.

Alpha-fetoprotein is a glycoprotein and various glycosylated forms of AFP have been described. Lectins can be used in the analysis of glycoproteins. By using the selective binding capacity of a lectin to the sugar chain structure of a glycoprotein it is possible to separate and concentrate the marker glycoprotein fraction(s) having a specific sugar chain structure. In the case of AFP, the lectin derived from Lens culinaris agglutinin-A (LCA) has been widely used. The Lens culinaris agglutinin (LCA)-reactive fraction of α-fetoprotein (AFP-L3) is specifically increased in patients with HCC. Many attempts have been made to specifically measure AFP-L3, e.g., by affinity electrophoresis using LCA, lectin-based ELISAs or by antibodies specifically binding the L3-form of AFP.

Protein induced by vitamin K absence/antagonist-II (PIVKA-II), also known as des-gamma-carboxy-prothrombin (DCP) is an abnormal form of prothrombin protein elevated in HCC patients and used as an alternative HCC biomarker individually or in combination with AFP. Prothrombin has 10 potential gamma-carboxylation sites and various forms of PIVKA-II with different levels of under-carboxylation are present in the circulation. Different assays for PIVKA-II may detect a different set of PIVKA-II-forms and the specificity/sensitivity of PIVKA-II might be variable depending on the assay used and of limited utility in the detection of early stage HCC. The term “antibody” as used herein includes monoclonal antibodies, polyclonal antibodies, multi-specific antibodies (e.g., bispecific antibodies), and antibody fragments so long as they exhibit the desired antigen-binding properties.

The term “specific binding” or “specifically binds” as used herein refers to a binding reaction wherein binding pair molecules exhibit a binding to each other under conditions where they do not significantly bind to other molecules.

The term “specific binding” or “specifically binds”, when referring to a protein or peptide as an antibody or binding agent, refers to a binding reaction wherein a binding agent binds to the corresponding target molecule with a K_{D} 10⁻⁷ M or less. The term “specific binding” or “specifically bind” preferably refers to a K_{D} of 10⁻⁸ M or less or even more preferred of 10-9 M or less for its target molecule. The term “specific” or “specifically” is used to indicate that other molecules present in the sample do not significantly bind to the binding agent specific for the target molecule. Preferably, the level of binding to a molecule other than the target molecule results in a binding affinity which is only 10% or less, more preferably only 5% or less of the affinity to the target molecule. In other words, preferably the K_{D} for the binding to the target molecule is at least 10-fold lower, more preferably at least 20-fold lower than for the binding to a non-target protein.

The methods and uses of the disclosure provide valuable information about the presence of HCC in a subject. In particular embodiments, the detection by the methods and uses of the invention is used to provide a treatment recommendation for the subject.

It will be appreciated that the methods and uses of the present disclosure can be carried out remotely from the subject or their attending physician, indeed, they could be carried out off-shore and the results communicated back. In particular embodiments, the results of the methods of the invention (e.g. determining whether or not a subject has HCC) are provided to a third party, such as the subject or their attending physician, a laboratory or health center.

Herein, the elements of the present invention are described. These elements are listed as aspects with specific embodiments, however, it should be understood that they may be combined in any manner and in any number to create additional aspects and embodiments. In particular, embodiments disclosed in context of one aspect apply mutatis mutandis to the other aspects. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

All references cited in this specification are herewith incorporated by reference with respect to their entire disclosure content and the disclosure content specifically mentioned in this specification.

The following examples and figures are provided to aid the understanding of the present invention, the true scope of which is set forth in the appended claims. It is understood that modifications can be made in the procedures set forth without departing from the spirit of the invention. Also provided is a description of amino acid sequences disclosed herein.

### Description of Sequences

SEQ ID NO: 1 shows the amino acid sequence of human haptoglobin; N184 is printed in bold and is underlined:

MSALGAVIAL LLWGQLFAVD SGNDVTDIAD DGCPKPPEIA
HGYVEHSVRY QCKNYYKLRT EGDGVYTLND KKQWINKAVG
DKLPECEADD GCPKPPEIAH GYVEHSVRYQ CKNYYKLRTE
GDGVYTLNNE KQWINKAVGD KLPECEAVCG KPKNPANPVQ
RILGGHLDAK GSFPWQAKMV SHHNLTTGAT LINEQWLLTT
AKNLFLNHSE NATAKDIAPT LTLYVGKKQL VEIEKVVLHP
NYSQVDIGLI KLKQKVSVNE RVMPICLPSK DYAEVGRVGY
VSGWGRNANF KFTDHLKYVM LPVADQDQCI RHYEGSTVPE
KKTPKSPVGV QPILNEHTFC AGMSKYQEDT CYGDAGSAFA
VHDLEEDTWY ATGILSEDKS CAVAEYGVYV KVTSIQDWVQ
KTIAEN

(sequence listing; Uniprot P00738, Version 221).

SEQ ID NO: 2 shows the amino acid sequence of the peptide part comprised in an exemplary glycopeptide for detecting glycan structures at position N184 (e.g. glycopeptide of formula 1); the sequence corresponds to amino acid positions 179 to 194 of SEQ ID NO:1 and the Asn residue corresponding to N184 of SEQ ID NO: 1 is printed in bold and is underlined:

MVSHHNLTTGATLINE

SEQ ID NO: 3 shows the amino acid sequence of the peptide comprised in an exemplary glycopeptide for detecting glycan structures at position N207; the Asn residue corresponding to N207 of SEQ ID NO: 3 is printed in bold and is underlined:

NLFLNHSE

SEQ ID NO: 4 shows the amino acid sequence of the peptide comprised in an exemplary glycopeptide for detecting glycan structures at position N211; the Asn residue corresponding to N211 of SEQ ID NO: 4 is printed in bold and is underlined: NATAK

NATAK

SEQ ID NO: 5 shows the amino acid sequence of the peptide comprised in an exemplary glycopeptide for detecting glycan structures at position N241; the Asn residue corresponding to N241 of SEQ ID NO: 5 is printed in bold and is underlined:

VVLHPNYSQVD

### DESCRIPTION OF FIGURES

FIG. 1: Schematic, representing various forms of haptoglobin: The three types of haptoglobin comprise (always together with the β-chain) either twice the α1-chain (1-1); al- and one α2-chain (2-1) or two α2-chains (2-2) of haptoglobin.

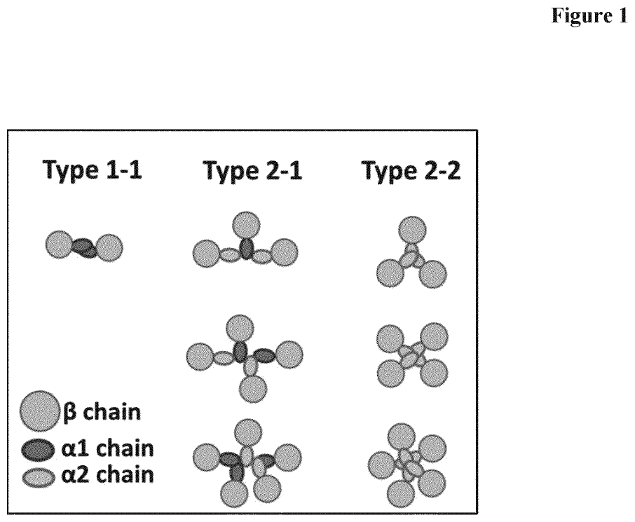

FIG. 2: Structure of Formula 1 (Comp. 41): HexNAc(5)Hex(6)Fuc(1)NeuAc(2) at position N-184. Left: glycan structure drawn by using the monosaccharide abbreviations following the SNFG (Symbol Nomenclature for Glycans) system (PMID 26543186, Glycobiology 25: 1323-1324, 2015) Connections are depicted by black lines. Right: Symbolic depiction of the glycan structure: Monosaccharide symbols follow the SNFG.

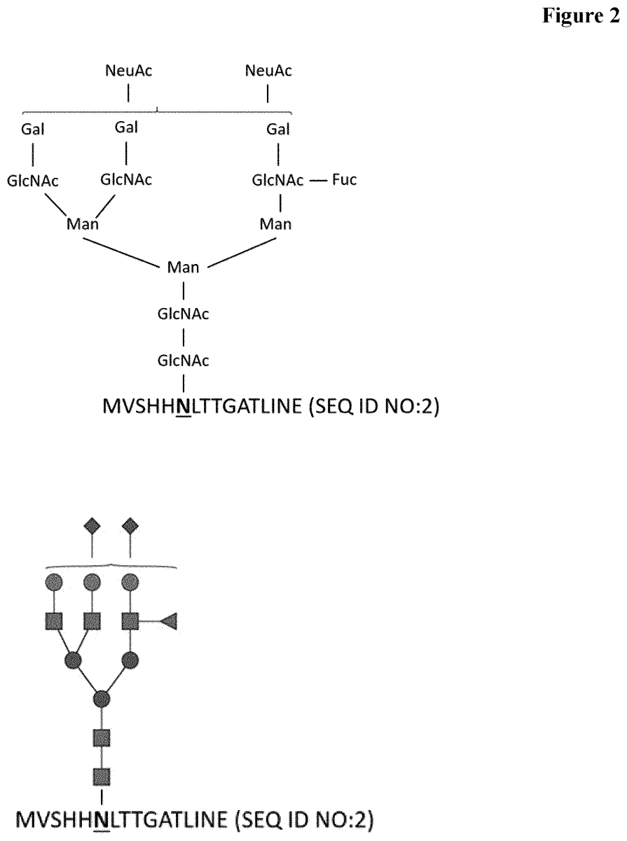

FIG. 3: Box-Blots for various glycopeptides at position N184 of the β-chain of haptoglobin (SEQ ID NO: 1) indicative for HCC: Site-specific glycan analysis revealed quite a few glycopeptides either elevated or decreased in HCC. The amounts of Hp glycopeptides were compared between controls, early and late stage HCC cohorts. Statistically significant differences between controls (C) early stage HCC (ESH) and late stage HCC (LSH) were determined by Wilcoxen (Mann-Whitney U) tests.

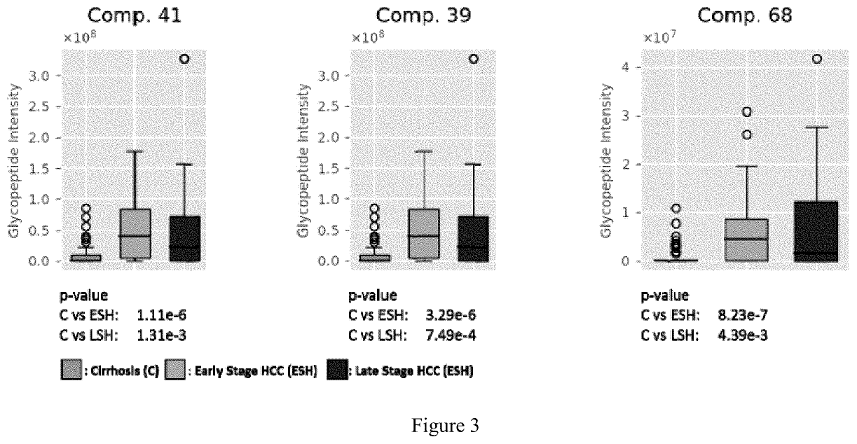

FIG. 4: Overview for upregulated glycopeptides on glycosylation site N-184. Monosaccharide symbols follow the SNFG (Symbol Nomenclature for Glycans) system (PMID 26543186, Glycobiology 25: 1323-1324, 2015). Only top 5 glycopeptides differentiating in early plus late stage HCC versus controls, are shown.

FIG. 5: Receiver Operator Curves (ROCs): for the compound HexNAc(5)Hex(6)Fuc(1)NeuAc(2) at position N-184 showing it's diagnostic performance for the detection of early and late HCC. The specificity and sensitivity values are for the sensitivity and specificity with 0.9 cutoff, respectively.

FIG. 6: Overview for up-/downregulated glycopeptides on all haptoglobin glycosylation sites (N-184, N-207, N-211 and N-241). Monosaccharide symbols follow the SNFG (Symbol Nomenclature for Glycans) system (PMID 26543186, Glycobiology 25: 1323-1324, 2015). On the left hand, Hp glycopeptides are listed differentiating between early cases of HCC and controls. In the middle column glycopeptides are shown that are present in both early and late stage HCC and in the right column those glycopeptides are shown that are differentiating in late stage HCC versus controls. Only glycopeptides with AUC >70% are shown. The direction of the expression change (decrease or increase vs. controls) is indicated by up-(regulated) or down-(regulated).

### EXAMPLES

### Example 1

### 1.1 Study Composition

EDTA-Plasma samples were obtained from 57 controls representing chronic liver diseases, incl. HBV, HCV and cirrhosis, and HCC patients including 33 individuals in early and 32 in late stage of HCC (see Table 1 for demographic information).

The stage classification of samples was based on Barcelona Liver Cancer (BCLC, Llovet J M, Brd C, Bruix J, Semin Liver Dis. 1999; 19(3):329-38) approach, with BCLC stages 0 and A classified as early stage HCC and stages B-D as late stage HCC. Following plasma preparation, the samples used in this analysis had been stored at −80° C. until analyzed as described below. Repeated freezing and thawing of samples had been avoided.

TABLE 1
Summary of demographic variables grouped
according to clinical status.

| | early stage | late stage | | | |
|---|---|---|---|---|---|
| | [symbol] | [symbol] | [symbol] | [symbol] | [symbol] |
| Age | | | | | |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| Sex | | | | | |
| [symbol] | [symbol] | [symbol] | [symbol] | [symbol] | [symbol] |
| [symbol] | [symbol] | [symbol] | [symbol] | [symbol] | [symbol] |
| Type | | | | | |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | [symbol] | [symbol] | [symbol] | [symbol] | [symbol] |
| [symbol] | | | | | |
| [symbol] | [symbol] | [symbol] | [symbol] | [symbol] | [symbol] |
| [symbol] | [symbol] | [symbol] | [symbol] | [symbol] | [symbol] |

[symbol]

indicates data missing or illegible when filed

1.2 Hp Immunoprecipitation from Plasma

In a first step immunocapture beads were prepared. 10 mg of streptavidine (SA)-coated latex beads were co-incubated on the rotator for 1 h at RT with 150 μg of biotin (Bi)-labeled F(ab′)2 fragment of a mouse monoclonal antibody against the Hp j-chain MAK<Haptoglobin>M-1.1.13-F(ab′)2-Bi (Roche Diagnostics GmbH, Mannheim, Germany). The co-incubation buffer used was phosphate buffered saline (PBS buffer: 10 mM phosphate buffer, 2.7 mM KCl, 137 mM NaCl, pH: 7.4). After coating of the F(ab′)2 fragments to the beads, the resulting coated beads were washed 3 times with PBS. Fifty microliters of each plasma sample was incubated with 5 μl of 20 mM DTT (dithiothreitol) for 1 h at 37° C. Then samples were treated with 10 μl of 50 mM IAM (iodoacetamide) for 30 min at RT. Thereafter, samples were diluted with 2 ml PBS and incubated together with MAK<Haptoglobin>M-1.1.13-F(ab′)2-Bi-SA-Beads (the antibody-coupled beads from the previous step). After 2 h incubation at RT on a rotator, unbound protein was washed away using two steps of washing with PBST (PBS buffer+0.1% Tween 20®) and two steps of PBS buffer. The Hp bound to the antibody-coated beads was eluted from the beads by incubating the beads in 500 μl of glycine buffer (0.2 M glycine; pH: 2.6) in two steps. The two step elution facilitated recovery. To naturalize the pH, 200 μl NaOH (1 N) was added to the eluate after each elution step.

### 1.3 FASP Digestion

The two eluted Hp fractions from the previous step were combined, loaded on a Nanosep® Centrifugal Devices with Omega™ Membrane—10K (PALL, US) filter (cut off: 10 kDa) and the sample centrifuged at 10.000 g for 20 min. In this step molecules, eg. proteins with a molecular weight of 10.000 or above are retained on the filter, while small molecules, e.g. salts pass through the filter and are removed. Then, 75 ng of Heavy Haptoglobin (recombinant Hp with isotopically labeled heavy Lys and Arg) protein was added to each sample/filter as an internal standard. Then 50 μl of denaturing buffer (1 mg/ml PPS (3-[3-(1,1-Bisheptyloxyethyl)pyridin-1-yl]propan-1-sulfonat) in 50 mM ammonium bicarbonate) and 5 μl of DTT (10 mM) were added to samples and the samples incubated at 50° C. for 30 min. In the next, step 5 μl of IAM (55 mM) was added to the samples and they were incubated for 30 min at 37° C. in the dark. Samples were centrifuged at 10.000 g for 20 min to wash out the buffer and washed once with 100 μl of ABC (ammonium bicarbonate, 50 mM) buffer. Then the protein retained on the filter was digested first by addition of 6 μg of trypsin in 50 μl of ABC buffer for 3 h at 37° C. Trypsin digestion was stopped by incubating the filters at 95° C. for 10 min. For the second digestion, 10 μg of GluC was added to the sample. For this second digestion the samples were incubated overnight at 25° C. Digested samples were eluted form the membrane by centrifugation at 10.000 g for 20 min.

### 1.4 LC-MS/MS Analysis

Twenty microliter of the peptides, obtained by enzymatic digestion as described in 1.3, were injected into the LC-MS/MS system (HF-X mass spectrometer (Thermo Fisher Scientific, Germany) coupled to a Vanquish (Thermo Fisher Scientific, Germany) UHPLC system). Peptides were separated at 50° C. by reverse phase chromatography on a C18 column (Waters, XSelect CSH C18 Column, 130 Å, 3.5 μm, 2.1 mm×150 mm). The LC had a flow rate of 320 μl/min and the gradient was set as follows: 0%-30% B (0-30 min), 30%-80% B (30-31 min), 80% B (31-36 min), 80%-0% B (36-37 min), and 0% B (37-42 min), wherein the eluents A and B were H₂O containing 0.1% formic acid, and acetonitrile containing 0.1% formic acid, respectively. Separated peptides were ionized by electrospray ionization (ESI) source and analyzed in the positive ion-mode and with data-dependent acquisition method. Full scan MS spectra were acquired in the range of 300-2000 m/z at a resolution of 60000, 10e6 automatic gain control (AGC), with 50 ms injection time. The top 5 most intense peaks from this scan, i.e. the survey scan, were selected for fragmentation with Higher-energy Collisional Dissociation (HCD), at a normalized collision energy of 28%, resolution of 15000, le5 AGC, with 150 ms injection time.

### 1.5 AFP and PIVKA-II Assays

AFP and PIVKA-II were measured using microchip capillary electrophoresis and a liquid-phase binding assay on the uTASWako i30 automated analyzer (Fujifilm Wako Pure Chemical Industries, Osaka, Japan), according to the instructions of the manufacturer.

### 1.6 Data Analysis

The acquired raw files from the mass spectrometer were processed by Byonic (Protein Metrics, CA, US) search engine embedded in Proteome Discoverer 2.2 (Thermo Fisher Scientific). The dataset was searched against Uniprot Haptoglobin protein sequence (P00738). For identification of glycopeptides, Byonic curated databases were used. The Byonic/Proteome Discoverer were configured as follows: the mass tolerance was set to 10 ppm for MS1 and 20 ppm or MS2. GluC and trypsin were set as proteases, allowing two miss cleavages. Carbamidomethylation of cysteine was set as fixed modification, and methionine oxidation and glycosylation on asparagine were specified as variable modifications. Results were filtered at 1% false discovery rate (FDR) and confidence threshold of the Byonic score >100. Composition of glycopeptides with significant difference in cohort groups and with AUC more than 0.7 were manually checked. In these cases, we checked the retention time, charge state, glycan oxonium ions and the isotopic pattern compared to the predicted isotopic pattern of the proposed glycopeptide. In the text we refer to a glycan composition as follows: HexNAc(x)Hex(x)Fuc(x)NeuAc(x), wherein the numbers in the parentheses represent the number of the respective monomer present in the glycan structure. HexNAc means N-acetylhexosamine, Hex means Hexose, Fuc means Fucose and NeuAc or Neu5Ac means N-Acetyl-neuraminic acid (sialic acid). Schematic representation is according to monosaccharide symbols follow the SNFG (Symbol Nomenclature for Glycans) system (PMID 26543186, Glycobiology 25: 1323-1324, 2015) details at NCBI (Ajit Varki et al., Symbol Nomenclature for Graphical Representations of Glycans, Glycobiology, Volume 25, Issue 12, December 2015, Pages 1323-1324, https://doi.org/10.1093/glycob/cwvO9l and Sriram Neelamegham et a.al, The SNFG Discussion Group, Updates to the Symbol Nomenclature for Glycans guidelines, Glycobiology, Volume 29, Issue 9, September 2019, Pages 620-624, https://doi.org/10.1093/glycob/cwzO45). Peak areas of glycopeptides XICs were automatically integrated by proteome discoverer and used as relative quantitative values.

### 1.7 Statistical Analysis

The abundance of glycopeptides in samples were normalized to abundance of top 3 peptides of spiked-in heavy Hp, to correct for possible handling, digestion or MS measurement variations. Missing values for a certain glycopeptide (i.e. glycopeptide below detection limit) were replaced by minimum amount value of that glycopeptide in the dataset. The significance of differences for a glycopeptide between the clinical groups was tested by calculating p-values, using the Wilcoxen (Mann-Whitney U) test.

In order to correct for multiple testing the Benjamini-Hochberg correction, with FDR control of 20% was used. To evaluate the diagnostic values of glycopeptides the Receiver-operating characteristic (ROC) curves were prepared and Area under the Curve (AUC) values were calculated by the DeLong method (Elisabeth R. DeLong, David M. DeLong and Daniel L. Clarke-Pearson (1988), Biometrics 44, 837-845) using R software (version 3.5.2) available at https://www.R-project.org.

### 2. Results

### 2.1 Glycan Performance for HCC Diagnosis

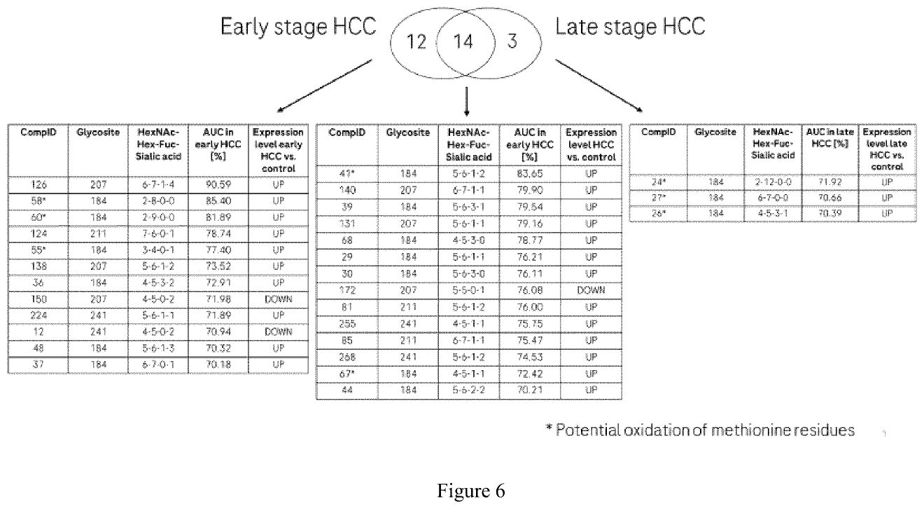

The overall analysis of glycan characteristics (e.g. fucosylation) provides an insight into the major changes in glycosylation pattern of Hp upon HCC progression. However, the types and the abundance of glycoforms varies at different Hp glycosites. It was therefore investigated whether Hp glycosylation site- and glycoform-specific analysis could be indicative of HCC. We compared the levels of site-specific glycopeptides between the controls, early and late stage HCC. Our observation revealed certain glycopeptides that are significantly upregulated in early stages of HCC compared to controls with an AUC of 0.70 or higher and that could be unambiguously assigned to a specific glycan structure (FIG. 6). This set of glycopeptides includes glycopeptides that are highly branched, fucosylated (compounds 126, 131 or 140) and highly mannosylated (compounds 58, 60, 24 and 27), and sialylated (compound 126).

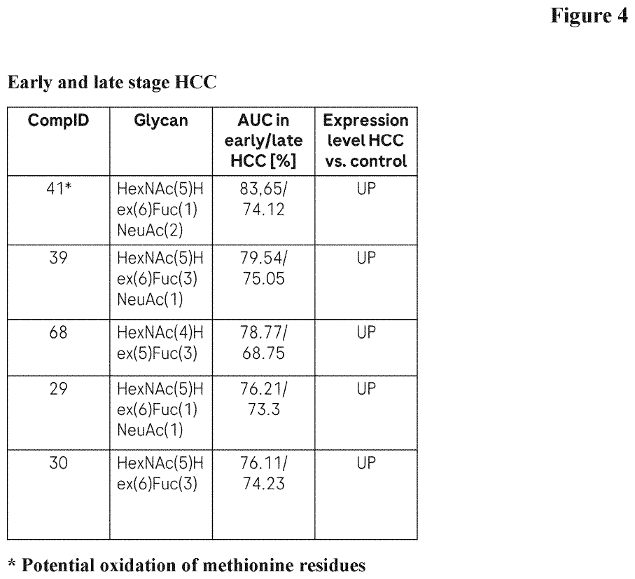

Besides, early stage HCC specific biomarkers, we have discovered fourteen other glycopeptides that were expressed significantly higher both in early and late stage HCC and that provided an AUC of 0.70 or higher in distinguishing early HCC patients and the cirrhotic controls (FIG. 6). Among these compound 41 showed the best AUC. These glycopeptides were expressed more as the HCC progressed from early to late stage. The top 5 Hp glycopeptides that were expressed significantly higher both in early and late stage HCC are listed in FIG. 4.

In addition to upregulated glycopeptides in HCC, we observed two glycopeptides that were significantly downregulated in early and late stage HCC compared to cirrhosis (FIG. 6), that were located at N207 glycosylation site. One of the glycoforms is a biantennary glycan with two sialic acids (HexNAc(4)Hex(5)NeuAc(2)), which is among the most abundant glycans. The other glycoform is a bisected biantennary glycan with one sialic acid (HexNAc(5)Hex(5)NeuAc(1)). These downregulated glycopeptides may be used for building a ratio with upregulated glycopeptides (e.g. compound 126 or compound 41) to further improve robustness by serving an internal control. Altogether, we have identified 29 glycan structures differentiating between controls and early- and late stages HCC with AUC above 70% (FIG. 6).

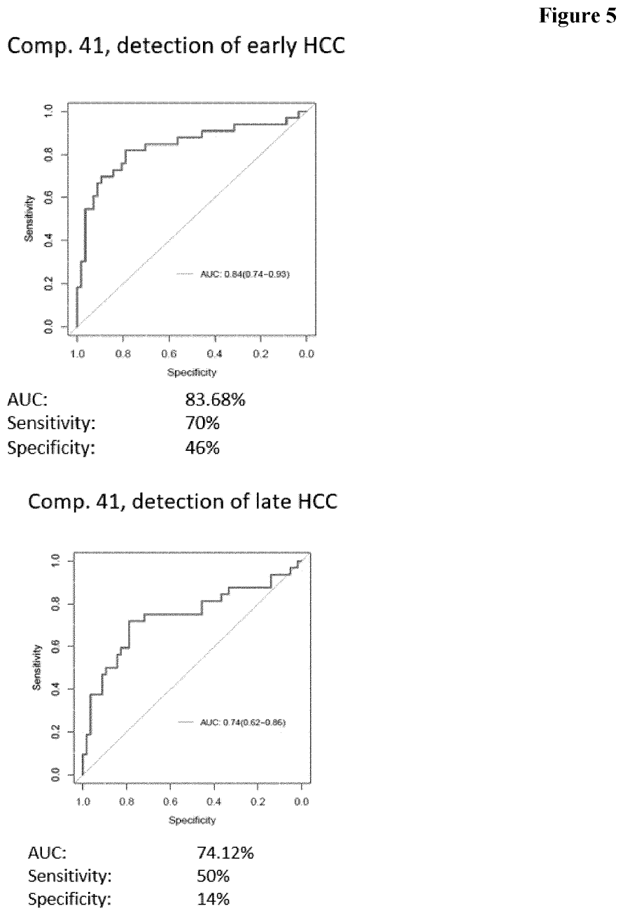

The by far best glycopeptide for detection early and late stage HCC was compound 41 on N184 (see FIGS. 4 and 5). Thus, among all glycolysation sites analyzed herein, Haptoglobin glycosylation at position N184 appears to be best suitable for detecting all stages of HCC. FIGS. 4 and 6 illustrate the data for all change glycopeptides using an AUC of 0.70 or greater as cut-off.

The results, glycan structures, peptide sequences and m/z values of the detected glycopeptides are summarized in Tables 2 and 3 below.

These results consistently revealed that glycopeptide analysis of Hp, especially at position N184 (but also at positions N207, N211 and N241) could provide glycobiomarkers that have better clinical values than established biomarkers for diagnosis of early stage HCC.

TABLE 2
Glycan structures, peptide sequences and m/z values of the detected glycopeptides significantly
differentiating between early- and late HCC vs. controls. AUC [%] for each examined group and
direction of expression change is indicated. *Potential oxidation of methionine residues

| | | | | | | Glyco- | |
|---|---|---|---|---|---|---|---|
| Compound | | Glyco- | | | | peptide | Glycan |
| ID | Peptide sequence | site | Glycan composition | m/z | Charge | mass | mass |
| Comp. 126 | NEFLNIHSE | N207 | HexNAc(6)Hex(7)Fuc(1)NeuAc(4) | 928.1685 | 5 | 4636.8124 | 3654.3059 |
| | (SEQ ID NO: 5) | | | | | | |
| Comp 58* | PAVSHHNEXTGATLINE | N184 | HexNAc(2)Hex(8) | 1152.8183 | 3 | 3456.4351 | 1702.5813 |
| | (SEQ ID NO: 2) | | | | | | |
| Comp. 41* | MAYSHANLITGATLINE | N184 | HexNAc(5)Hex(6)Fuc(2)NeuAc(2) | 1118.2098 | 4 | 4469.8163 | 2715.9625 |
| | (SEQ ID NO: 21 | | | | | | |
| Comp. 60* | MYSHHNLE GATLINE | N184 | HexNAc(2)Hex(9) | 1206.8562 | 3 | 3618.4942 | 1864.6342 |
| | (SEQ ID NO: 2) | | | | | | |
| Comp. 39 | MVSHARETIGATBINE | N184 | HexNAc(5)Hex(6)Fuc(3)NeuAc(1) | 1490.6144 | 3 | 4470.8367 | 2716.9829 |
| | (SEQ ID NO: 2) | | | | | | |
| Comp. 68 | MUSHHRLTIGATLINE | N184 | HexNAc(4)Hex(5)Fuc(3) | 950.1543 | 4 | 5797.5953 | 2059.7349 |
| | (SEQ ID NO: 2) | | | | | | |
| Comp. 124 | NATAK | N211 | HexNAc(6)Hex(7)NeuAc(1) | 1050.0805 | 3 | 3148.2191 | 2643.9414 |
| | (SEQ ID NO: 2) | | | | | | |
| Comp. 55* | MYSHANLTIGATLINE | N184 | (HexNAc(3)Hex(4)NeuAc(1) | 1101.4693 | 3 | 3302.3982 | 1548.5448 |
| | (SEQ ID NO: 2) | | | | | | |
| Comp. 140 | NEFLNHSE | N207 | HexNAc(6)Hex(7)Fuc(1}NeuAc(1) | 1255.1616 | 3 | 3763.4701 | 2789,9998 |
| | (SEQ ID NO: 3} | | | | | | |
| Comp. 29 | PAYSHHNLITGATLINE | N184 | HexNAc(S)Hex(6)Fuc{1}NeuAc(1) | 1588.249 | 3 | 4162.7259 | 2424.8671 |
| | (SEQ ID NO: 2) | | | | | | |
| Comp. 30 | MVSHHNLTTGATENE | N184 | HexNAc(5)Hex(6)Fuc(8) | 1388.249 | 3 | 4162.7323 | 2425.8875 |
| | (SEQ ID NO: 2) | | | | | | |
| Comp. 81 | NATAK | N211 | HexNAc(S)Hex(6)Fuc(1}NeuAc(2) | 1074.0856 | 3 | 3220.2402 | 2715.9625 |
| | (SEQ ID NO: 4} | | | | | | |
| Comp 255 | VVLHPNYSQVD | N241 | HexNAc(4)Hex(5)Fuc(1)NeuAc(1) | 1110.7993 | 3 | 3330.3776 | 2059.7349 |
| | (SEQ ID NO: 5) | | | | | | |

| | AUC in | AUC in | Expression |
|---|---|---|---|
| Compound | early HCC | Late HCC | in HCC |
| ID | [%] | [%] | vs. Control |
| Comp. 126 | 90.59 | not | Higher |
| | | significant | |
| Comp 58* | 85.40 | not | Higher |
| | | significant | |
| Comp. 41* | 83.65 | 74.12 | Higher |
| Comp. 60* | 81.89 | not | Higher |
| | | significant | |
| Comp. 39 | 79.54 | 75.05 | Higher |
| Comp. 68 | 78.77 | 68.75 | Higher |
| Comp. 124 | 78.74 | not | Higher |
| | | significant | |
| Comp. 55* | 77.40 | not | Higher |
| | | significant | |
| Comp. 140 | 79.9 | 74.55 | Higher |
| Comp. 29 | 76.21 | 73.3 | Higher |
| Comp. 30 | 76.11 | 74.23 | Higher |
| Comp. 81 | 76.00 | 66.17 | Higher |
| Comp 255 | 75.75 | 65.75 | Higher |

TABLE 3
Glycan structures, peptide sequences and m/z values of the detected
glycopeptides significantly differentiating between early- and late HCC vs. controls.
AUC [%] for each examined group and direction of expression change is indicated.
*Potential oxidation of methionine residues.

| | | | | | | Glyco- | |
|---|---|---|---|---|---|---|---|
| Compound | | Glyco- | | | | peptide | Glycan |
| ID | Peptide sequence | site | Glycan composition | m/z | Charge | mass | mass |
| Comp.131 | NLFLNHSE | N207 | HexNAc(5)Hex(6)Fuc(1)NeuAc(1) | 1699.68 | 2 | 3398.3409 | 2424.8671 |
| | (SEQ ID NO: 3) | | | | | | |
| Comp. 85 | NATAK | N211 | HexNAc(6)Hex(7)Fuc(1)NeuAc(1) | 1098.765 | 3 | 3294.2795 | 2789.9993 |
| | (SEQ ID NO: 4) | | | | | | |
| Comp. 150 | NLFLNHSE | N207 | HexNAc(4)Hex(5}NeuAc(2) | 1060.088 | 3 | 3178.2503 | 2204.7724 |
| | (SEQ ID NO: 3) | | | | | | |
| Comp. 172 | NLFLNHSE | N207 | HexNAc(5)Hex(5)NeuAc(2) | 982.0654 | 3 | 2944.1722 | 2116.7564 |
| | (SEQ ID NO: 3) | | | | | | |
| Comp. 268 | VVLHPNVSQVD | N241 | HexNAc(5)Hex(6)Fuc(1)NeuAc(2) | 1504.33 | 3 | 4510.9762 | 2715.9625 |
| | (SEQ ID NO: 5) | | | | | | |
| Comp. 35 | MVSHHNLTTGATLINE | N184 | HexNAc(4)Hex(5)Fuc(3)NeuAc(2) | 1096.204 | 4 | 4380.805 | 2642.9462 |
| | (SEQ ID NO: 2) | | | | | | |
| Comp. 67* | MVSHHNLTTGATLINE | N184 | HexNAc(4)Hex(5)Fuc(1)NeuAc(1) | 1271.872 | 3 | 3813.6022 | 2059.7349 |
| | {SEQ ID NO: 2) | | | | | | |
| Comp. 224 | VVLHPNYSQVD | N241 | HexNAc(5)Hex(6)Fuc(1)NeuAc(1) | 1232.507 | 3 | 3695.5098 | 2424.8671 |
| | (SEQ ID NO: 5] | | | | | | |
| Comp. 12 | VVLHPNYSQVD | N241 | HexNAc(4)Hex(5}NeuAc(2) | 1201.843 | 3 | 3603.5136 | 2204.7724 |
| | (SEQ ID NO: 5} | | | | | | |
| Comp. 48 | MVSHHNLTTGATLINE | N184 | HexNAc(5)Hex(6)Fuc(1)NeuAc(3) | 950.1543 | 4 | 3797.5953 | 2059.7349 |
| | (SEQ ID NO: 2) | | | | | | |
| Comp, 44 | MVSHHNLTTGATLINE | N184 | HexNAc(5)Hex(6)Fuc(2)NeuAc(2) | 1150.474 | 4 | 4598.8748 | 2862.0205 |
| | (SEQ ID NO: 2) | | | | | | |
| Comp. 37 | MVSHHNLTTGATLINE | N184 | HexNAc(6)Hex(7)NeuAc(1) | 1461.272 | 3 | 4381.8015 | 2644.9618 |
| | (SEQ ID NO: 2) | | | | | | |
| Comp. 138 | NLFLNHSE | N207 | HexNAc(5)Hex(6)Fuc(1)NeuAc(2) | 1230.484 | 3 | 3689.4373 | 2715.9625 |
| | (SEQ ID NO: 3) | | | | | | |

| | AUC in | AUC in | Expression |
|---|---|---|---|
| Compound | early HCC | Late HCC | in HCC |
| ID | [%] | [%] | vs. Control |
| Comp.131 | 79.16 | 69.35 | Higher |
| Comp. 85 | 75.47368 | 69.4 | Higher |
| Comp. 150 | 71.98 | 68.09 | Lower |
| Comp. 172 | 76.08 | 71.33 | Lower |
| Comp. 268 | 74.52632 | 68.42 | Higher |
| Comp. 35 | 72.91228 | not | Higher |
| | | significant | |
| Comp. 67* | 72.42105 | 66 | Higher |
| Comp. 224 | 71.89474 | not | Higher |
| | | significant | |
| Comp. 12 | 70.94 | not | Lower |
| | | significant | |
| Comp. 48 | 70.32 | not | Higher |
| | | significant | |
| Comp, 44 | 70.21053 | 71.82 | Higher |
| Comp. 37 | 70.17544 | not | Higher |
| | | significant | |
| Comp. 138 | 73.52 | 66 | Higher |

### 2.2 Top Glycans for Detection of (Early Stage or Early and Late Stage) HCC

The results show that in particular the Hp glycopeptide Compound 41 (i.e. HexNAc(5)Hex(6)Fuc(1)NeuAc(2) at position N184) has a high potential to be of clinical utility in the detection of HCC, especially as in the detection of HCC at an early stage. These is obvious from the values for the area under the curve (AUC) given for each of these glycopeptides. It is striking to see that for this glycopeptide the calculated AUC has exceeded the AUC value of AFP and that glycopeptide improves performance of each individual protein biomarker and their combination. Other Hp glycopeptide compounds with a good diagnostic potential for early HCC are shown in Table 2 and FIG. 6.

Moreover, a number of glycans with a good performance in identifying early HCC and late HCC cases were identified. In particular, compound 41 has a high potential of clinical utility. This Hp glycopeptide is the one with the best AUC for early HCC, which also shows a significant AUC for late HCC. Other Hp glycopeptide compounds with a good diagnostic potential for early and late HCC are shown in Table 2 and FIGS. 4 and 6.

### 2.3 Marker Combinations

To analyse the added value of the compound 41 to PIVKA-II and/or AFP, logistic regression models were build, which consisted of compound 41, PIVKA-II and/or AFP. In these models logarithmic transformations were applied to the markers to reduce skewness of the marker distributions. For all compounds, these multivariate models were constructed and the performance in form of the combined AUC was compared. The marker combinations are summarized in Table 4. Other models besides of logistic regression (or logistic regression models with interaction terms between the variables) could not improve the performance of the aforementioned logistic regression models significantly.

TABLE 4
Combination of glycopeptide compound 41 improves the clinical value of AFP.
The AUC value for AFP for early diagnosis of HCC in our cohort is 83%.

| | | | | | Multivariate |
|---|---|---|---|---|---|
| | | | Multivariate | Multivariate | AUC [%] |
| | | Univariate | AUC [%] | AUC [%] | with AFP |
| Compound ID | m/z | AUC [%] | with AFP | with PIVKA-II | and PIVKA-II |
| 41 | 1118.2098 | 84 (74-93) | 90 (83-96) | 87 (79-96) | 93 (87-99) |
| AFP | — | 83 (75-92) | — | 94 (89-99) | — |
| PIVKA-II | — | 88 (80-97) | 94 (89-99) | — | — |

Combining these two biomarkers improves the AUC to 87%, combination of glycopeptide with AFP and PIVKA-II improves the AUC to 93%.

1. An in vitro method for aiding in the detection of hepatocellular carcinoma (HCC) in a subject comprising the steps of:
a) determining the amount of the glycan structure HexNAc(5)Hex(6)Fuc(1)NeuAc(2) at position N184 of haptoglobin (i.e. of the β-chain of haptoglobin having the sequence given in SEQ ID NO: 1) in a sample obtained from said subject; and
b) (i) comparing the amount of said N-glycan structure determined in a) to a reference amount of said N-glycan structure, wherein an increased amount of HexNAc(5)Hex(6)Fuc(1)NeuAc(2) at position 184 of haptoglobin in said subject's sample relative to the reference amount of this N-glycan structure is indicative for HCC; or (ii) determining a score for detecting HCC taking into account the amount HexNAc(5)Hex(6)Fuc(1)NeuAc(2) at position N184 of haptoglobin determined in a) and comparing the determined score for detecting HCC to a reference value for said score indicative for HCC.

2. An isolated glycopeptide having a peptide part and a N-glycan part, wherein the peptide part comprises or consists of the amino acid sequence MVSHHNLTTGATLINE (SEQ ID NO: 2) and wherein the N-glycan part is HexNAc(5)Hex(6)Fuc(1)NeuAc(2), and wherein the N-glycan part is attached to the N in position 6 of SEQ ID NO: 2.

3. The glycopeptide of claim 2, wherein the glycopeptide has the structure as shown in Formula 1

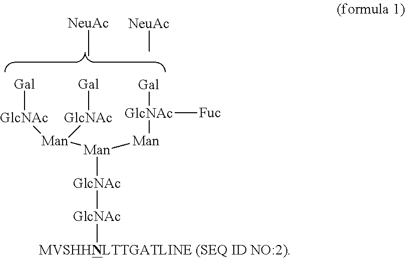

4. (canceled)

5. The method of claim 1, wherein the method further comprises determining the amount of AFP in the sample or another sample from the same subject and wherein the score for detecting HCC takes into account the determined amount of AFP.

6. The method of claim 1, wherein the haptoglobin is hydrolyzed to glycopeptides, said glycopeptides having a peptide part and a N-glycan part, wherein the peptide part comprises or consists of the amino acid sequence MVSHHNLTTGATLINE (SEQ ID NO: 2) and wherein the N-glycan part is HexNAc(5)Hex(6)Fuc(1)NeuAc(2), and wherein the N-glycan part is attached to the N in position 6 of SEQ ID NO: 2,
and wherein the amount of the glycan structure HexNAc(5)Hex(6)Fuc(1)NeuAc(2) at position N184 of haptoglobin is determined by determining the amount of the glycopeptide or corresponds to the amount of the glycopeptide.

7. The method of claim 6, wherein the method comprises purifying haptoglobin from the sample before the haptoglobin is hydrolyzed.

8. The method of claim 1, wherein the amount of the glycan structure HexNAc(5)Hex(6)Fuc(1)NeuAc(2) at position N184 of haptoglobin is determined using mass spectrometry.

9. A method for detecting and/or quantifying the glycopeptide according to claim 2, the method comprising the steps of:
a) purifying haptoglobin from a sample to be analyzed;
b) digesting the haptoglobin obtained in step a) by GluC and trypsin; and
c) detecting the glycopeptides obtained in b), thereby detecting the glycopeptide according to claim 2.

10. A computer-implemented method for aiding in the detection of hepatocellular carcinoma (HCC) in a subject, said method comprising:
a) receiving data comprising the amount of the glycan structure HexNAc(5)Hex(6)Fuc(1)NeuAc(2) at position N184 of haptoglobin (i.e. of the β-chain of haptoglobin having the sequence given in SEQ ID NO: 1) in a sample obtained from said subject;
b) i) comparing the amount of HexNAc(5)Hex(6)Fuc(1)NeuAc(2) received in a) to a reference amount of said glycan structure, wherein an increased amount of HexNAc(5)Hex(6)Fuc(1)NeuAc(2) at position N184 of haptoglobin in said subject's sample relative to the reference amount of this glycan structure is indicative for HCC, or (ii) calculating a score for detecting HCC taking into account the amount of HexNAc(5)Hex(6)Fuc(1)NeuAc(2) at position N184 of haptoglobin received in a) and comparing the calculated score for detecting HCC to a reference value for said score indicative for HCC; and
c) aid in determining whether the subject has HCC based the comparison in b).

11. The computer-implemented method of claim 10, wherein the method further comprises receiving data comprising the amount of AFP in the sample or another sample from the same subject and wherein the calculation of the score for detecting HCC in c) further takes into account the determined amount of AFP.

12. (canceled)

13. The method of claim 1, wherein HCC is early HCC.

14. The method of claim 1, wherein the sample is a blood sample.

15. The method of claim 14, wherein the sample is a serum or plasma.
